(19) United States
(12) Patent Application Publication
(10) Pub. No.: US 2016/0206608 A1
(43) Pub. Date: Jul. 21, 2016

(54) CRIZOTINIB FOR USE IN THE TREATMENT OF CANCER

(71) Applicants: James Gail Christensen, San Diego, CA (US); Yahong Zou, San Diego, CA (US)

(72) Inventors: James Gail Christensen, San Diego, CA (US); Yahong Zou, San Diego, CA (US)

(73) Assignee: Pfizer Inc., New York, NY (US)

(21) Appl. No.: 14/236,034

(22) PCT Filed: Jul. 24, 2012
(86) PCT No.: PCT/IB2012/053765

§ 371 Date: Sep. 28, 2015

Related U.S. Application Data
(60) Provisional application No. 61/514,386, filed on Aug. 2, 2011.

(51) Int. Cl. A61K 31/4545

(52) U.S. Cl. CPC A61K 31/4545

(57) ABSTRACT

The present invention relates to the use of ROS kinase inhibitors for treating abnormal cell growth in mammals. In particular, the invention provides methods of treating mammals suffering from cancer mediated by at least one genetically altered ROS. In particular, the invention provides methods of treating mammals suffering from cancer mediated by at least one genetically altered ROS by administration of crizotinib.

This application claims the benefit of U.S. Provisional Application No. 61/514,386 filed on Aug. 2, 2011, the contents of which are hereby incorporated by reference in their entirety.

### FIELD OF THE INVENTION

The present invention relates to the use of ROS inhibitors for treating abnormal cell growth in mammals. In particular, the invention provides methods of treating mammals suffering from cancer.

### BACKGROUND OF THE INVENTION

Human cancers comprise a diverse array of diseases that collectively are one of the leading causes of death in developed countries throughout the world (American Cancer Society, Cancer Facts and Figures 2005. Atlanta: American Cancer Society; 2005). The progression of cancers is caused by a complex series of multiple genetic and molecular events including gene mutations, chromosomal translocations, and karyotypic abnormalities (Hanahan et al. Cell 100:57-70 (2000)). Although the underlying genetic causes of cancer are both diverse and complex, each cancer type has been observed to exhibit common traits and acquired capabilities that facilitate its progression. These acquired capabilities include dysregulated cell growth, sustained ability to recruit blood vessels (i.e., angiogenesis), and ability of tumor cells to spread locally as well as metastasize to secondary organ sites (Hanahan et al. Cell (2000)). Therefore, the ability to identify novel therapeutic agents that 1) inhibit molecular targets that are altered during cancer progression or 2) target multiple processes that are common to cancer progression in a variety of tumors presents a significant unmet need.

V-ros UR2 sarcoma virus oncogene homolog 1 (ROS-1 or ROS) is a proto-oncogene receptor tyrosine kinase that belongs to the insulin receptor subfamily, and is involved in cell proliferation and differentiation processes. Nagarajan et al. Proc Natl Acad Sci 83:6568-6572 (1986)). ROS is expressed, in humans, in epithelial cells of a variety of different tissues. Defects in ROS expression and/or activation have been found in glioblastoma, as well as tumors of the central nervous system (Charest et al., Genes Chromos. Can. 37(1): 58-71 (2003)). Genetic alterations involving ROS that result in aberrant fusion proteins of ROS kinase have been described, including the FIG-ROS deletion translocation in glioblastoma (Charest et al. (2003); Birchmeier et al. Proc Natl Acad Sci 84:9270-9274 (1987)) and NSCLC (Rimkunas et al. Clin Cancer Res epub, June 1. (2012)), the SLC34A2-ROS translocation in NSCLC (Rikova et al. Cell 131:1190-1203 (2007), the CD74-ROS translocation in NSCLC (Rikova et al. (2007)) and cholangiocarcinoma (Gu et al. PLoS ONE 6(1): e15640 (2011)), and a truncated, active form of ROS known to drive tumor growth in mice (Birchmeier et al. Mol. Cell. Bio. 6(9):3109-3115 (1986)). Additional fusions, including TPM3-ROS1, SDC4-ROS1, EZR-ROS1 and LRIG3-ROS1, have been reported in lung cancer patient tumor samples (Takeuchi et al. Nature Medicine (2012)).

Sodium Dependent Phosphate Transporter Isoform NaPi-3b protein (SLC34A2) is a 690 amino acid phosphate transporter protein that is expressed in human lung and small intestine, and which has sodium-dependent activity. Defects in SLC34A2 expression and/or activity have been found in ovarian cancer (Rangel et al., Oncogene 22(46): 7225-7232 (2003)). CD74 is an integral membrane protein that functions as a MHC class II chaperone protein with high affinity for the MIF immune cytokine (Leng et al. J. Exp. Med. 197:1467-1476 (2003). FIG (Fused in Glioblastoma) is a gene that encodes for a 454-amino acid protein that includes a PSD-95, Disc Large, ZO-1 (PDZ) domain, two coiled coil regions, and a leucine zipper. FIG has been shown to associate peripherally with the Golgi apparatus by interacting through its second coiled coil domain with a SNARE protein, and has therefore been postulated to play a role in Golgi-mediated vesicular transport (Charest et al. (2003).

The SLC34A2-ROS translocation occurs between chromosome (4p15) and chromosome (6q22) and produces two fusion protein variants that combine the N-terminus of Sodium-Dependent Phosphate Transporter Isoform NaPi-3b protein (SLC34A2), with the transmembrane and kinase domains of Proto-Oncogene Tyrosine Protein Kinase ROS precursor (ROS) kinase (WO 2007/084631). To date, two variants of SLC34A2-ROS fusion proteins, which are 724 amino acids (SLC34A2-ROS(L); long variant) and 621 amino acids (SLC34A2-ROS(S); short variant), respectively, have been identified (WO 2007/084631). The SLC34A2-ROS translocation can also be described as a fusion of the ROS gene and the SLC34A2 gene which subsequently produces an aberrant SLC34A2-ROS fusion protein characterized by a protein sequence encoded by the SLC34A2-ROS fusion gene.

The CD74-ROS translocation occurs between chromosome (5q32) and chromosome (6q22) and produces a fusion protein that combines the N-terminus of CD74, with the transmembrane and kinase domains of Proto-Oncogene Tyrosine Protein Kinase ROS precursor (ROS) kinase. The resulting CD74-ROS fusion protein is a 703 amino acid protein (WO 2009/051846). The CD74-ROS translocation can also be described as a fusion of the ROS gene and the CD74 gene which subsequently produces an aberrant CD74-ROS fusion protein characterized by a protein sequence encoded by the CD74-ROS fusion gene.

The FIG-ROS deletion translocation occurs by way of an intra-chromosomal homozygous deletion of 240 kilobases on chromosome (6q21) to produce a constitutively activated tyrosine kinase (Charest et al. (2003)). Variants of FIG-ROS fusion proteins, which are 878 amino acids (FIG-ROS(L); long variant) and 630 amino acids (FIG-ROS(S); short variant), respectively, have been reported (Gu et al. (2011); US 2011/0287445). Because fusions and deletions involving the ROS gene have been implicated in the etiology of human cancers, finding inhibitors of ROS that can function to attenuate the activity of ROS kinase activity in such fusions and deletions represents a significant unmet need in cancer therapy.

### SUMMARY OF THE INVENTION

In one aspect the present invention provides as method of treating cancer in a human in need of such treatment comprising, administering to said human a therapeutically effective amount of a ROS kinase inhibitor of the formula 1:

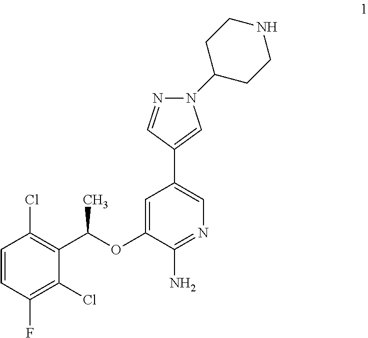 [SMILES: C[C@@H](Oc1cc(-c2cnn(C3CCNCC3)c2)cnc1N)c1c(Cl)ccc(F)c1Cl]

or a pharmaceutically acceptable salt thereof, wherein the cancer is mediated by at least one genetically altered ROS. The compound of formula 1 may be variously referred to herein by its generic name, crizotinib, or by its chemical name, 3-[(R)-1-(2,6-dichloro-3-fluoro-phenyl)-ethoxy]-5-(1-piperidin-4-yl-1H-pyrazol-4-yl)-pyridin-2-ylamine.

In one embodiment of this aspect of the invention, the at least one genetically altered ROS is a fusion gene of ROS. In another embodiment of this aspect, the fusion gene of ROS is SLC34A2-ROS gene or CD74-ROS gene. In another embodiment of this aspect, the at least one genetically altered ROS is a genetic deletion involving ROS kinase. In another embodiment of this aspect, the genetic deletion is FIG-ROS gene. In another embodiment of this aspect, the at least one genetically altered ROS is a genetically altered ROS kinase. In another embodiment of this aspect, the genetically altered ROS kinase is a ROS fusion. In another embodiment of this aspect, the ROS fusion is SLC34A2-ROS kinase or CD74-ROS kinase. In another embodiment of this aspect, the at least one genetically altered ROS is a deletion protein involving ROS kinase. In another embodiment of this aspect, the deletion protein is FIG-ROS kinase.

In another embodiment of this aspect, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof. In another embodiment of this aspect, the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In another embodiment of this aspect, the cancer is non-small cell lung cancer (NSCLC). In another embodiment of this aspect, the cancer is glioblastoma. In another embodiment of this aspect, the compound of the formula 1 is administered as a pharmaceutical composition comprising the compound of the formula 1 and at least one pharmaceutically acceptable carrier. In another embodiment of this aspect, the compound of the formula 1 or a pharmaceutically acceptable salt thereof is administered as a pharmaceutical composition comprising the compound of formula 1 or a pharmaceutically acceptable salt thereof and at least one pharmaceutically acceptable carrier.

In another aspect the present invention provides a method comprising administering to a mammal having an abnormal cell growth mediated by ROS kinase a therapeutically effective amount of a ROS kinase inhibitor. In one embodiment of this aspect of the invention, the abnormal cell growth is mediated by at least one genetically altered ROS kinase. In another embodiment, the abnormal cell growth is mediated by a fusion gene of ROS kinase. In another embodiment, the abnormal cell growth is mediated by a genetic deletion involving ROS kinase. In another embodiment, the fusion gene is SLC34A2-ROS or CD74-ROS. In another embodiment, the genetic deletion is FIG-ROS. In another embodiment, the abnormal cell growth is mediated by a fusion protein of ROS kinase. In another embodiment, the abnormal cell growth is mediated by a deletion protein involving ROS kinase. In another embodiment, the fusion protein is SLC34A2-ROS or CD74-ROS. In another embodiment, the deletion protein is FIG-ROS. In some such embodiments of this aspect, the method comprises administering to said mammal having an abnormal cell growth mediated by ROS kinase a therapeutically effective amount of a ROS kinase inhibitor, thereby treating said abnormal cell growth.

In another embodiment of this aspect, the ROS kinase inhibitor is a small molecule inhibitor of ROS kinase. In another embodiment, the ROS kinase inhibitor is an amino-pyridine compound or an amino-pyrazine compound.

In another embodiment of each of the preceding aspects of the invention, the ROS kinase inhibitor is a compound of the formula 1:

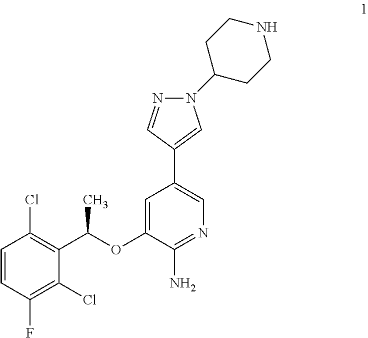 [SMILES: C[C@@H](Oc1cc(-c2cnn(C3CCNCC3)c2)cnc1N)c1c(Cl)ccc(F)c1Cl]

or a pharmaceutically acceptable salt thereof.

In another embodiment of this aspect of the invention, the abnormal cell growth is cancer. In another embodiment of each of the preceding aspects of the invention, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In yet another embodiment of this aspect of the invention, the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In yet another embodiment of each of the preceding aspects of the invention, the cancer is non-small cell lung cancer (NSCLC). In yet another embodiment, the cancer is glioblastoma.

In yet another embodiment of this aspect of the invention, the compound or pharmaceutically acceptable salt thereof is administered as a pharmaceutical composition comprising the compound of the formula 1 and at least one pharmaceutically acceptable carrier. In another embodiment of this aspect, the compound of the formula 1 or a pharmaceutically acceptable salt thereof is administered as a pharmaceutical composition comprising the compound of formula 1 or a pharmaceutically acceptable salt thereof and at least one pharmaceutically acceptable carrier.

In another aspect the present invention provides a method comprising administering to a mammal having cancer mediated by ROS kinase a therapeutically effective amount of a ROS kinase inhibitor. In one embodiment of this aspect of the invention, the cancer is mediated by at least one genetically altered ROS kinase. In another embodiment, the cancer is mediated by a fusion gene of ROS kinase. In another embodiment, the abnormal cell growth is mediated by a genetic deletion involving ROS kinase. In another embodiment, the fusion gene is SLC34A2-ROS or CD74-ROS. In another embodiment, the genetic deletion is FIG-ROS. In another embodiment, the abnormal cell growth is mediated by a fusion protein of ROS kinase. In another embodiment, the abnormal cell growth is mediated by a deletion protein involving ROS kinase. In another embodiment, the fusion protein is SLC34A2-ROS or CD74-ROS. In another embodiment, the deletion protein is FIG-ROS. In some such embodiments of this aspect, the method comprises administering to said mammal having cancer mediated by ROS kinase a therapeutically effective amount of a ROS kinase inhibitor, thereby treating said cancer.

In another embodiment of this aspect, the ROS kinase inhibitor is a small molecule inhibitor of ROS kinase. In another embodiment, the ROS kinase inhibitor is an amino-pyridine compound or an amino-pyrazine compound. In another embodiment of each of the preceding aspects of the invention, the ROS kinase inhibitor is a compound of the formula 1:

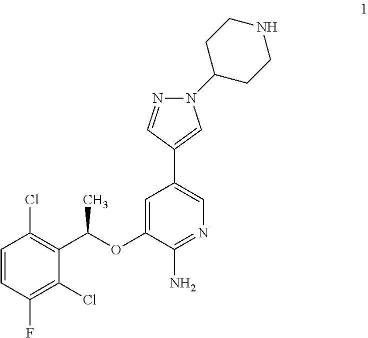 [SMILES: C[C@@H](Oc1cc(-c2cnn(C3CCNCC3)c2)cnc1N)c1c(Cl)ccc(F)c1Cl]

or a pharmaceutically acceptable salt thereof.

In another embodiment, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In yet another embodiment of this aspect of the invention, the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In yet another embodiment, the cancer is non-small cell lung cancer (NSCLC). In yet another embodiment, the cancer is glioblastoma.

In yet another embodiment of this aspect of the invention, the compound or pharmaceutically acceptable salt thereof is administered as a pharmaceutical composition comprising the compound of the formula 1 and at least one pharmaceutically acceptable carrier. In another embodiment of this aspect, the compound of the formula 1 or a pharmaceutically acceptable salt thereof is administered as a pharmaceutical composition comprising the compound of formula 1 or a pharmaceutically acceptable salt thereof and at least one pharmaceutically acceptable carrier.

In another aspect the present invention provides a method comprising treating cancer mediated by at least one ROS kinase in a mammal in need of such treatment by administering a therapeutically effective amount of a ROS kinase inhibitor. In one embodiment of this aspect of the invention, the cancer is mediated by at least one genetically altered ROS kinase. In another embodiment, the cancer is mediated by a fusion gene of ROS kinase. In another embodiment, the cancer is mediated by a genetic deletion involving ROS kinase. In another embodiment, the fusion gene is SLC34A2-ROS or CD74-ROS. In another embodiment, the genetic deletion is FIG-ROS. In another embodiment, the abnormal cell growth is mediated by a fusion protein of ROS kinase. In another embodiment, the abnormal cell growth is mediated by a deletion protein involving ROS kinase. In another embodiment, the fusion protein is SLC34A2-ROS or CD74-ROS. In another embodiment, the deletion protein is FIG-ROS.

In another embodiment of this aspect, the ROS kinase inhibitor is a small molecule inhibitor of ROS kinase. In another embodiment, the ROS kinase inhibitor is an amino-pyridine compound or an amino-pyrazine compound. In another embodiment of each of the preceding aspects of the invention, the ROS kinase inhibitor is a compound of the formula 1:

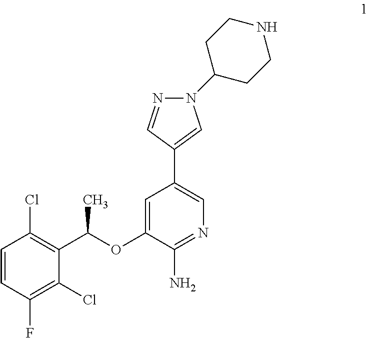 [SMILES: C[C@@H](Oc1cc(-c2cnn(C3CCNCC3)c2)cnc1N)c1c(Cl)ccc(F)c1Cl]

or a pharmaceutically acceptable salt thereof.

In another embodiment, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In yet another embodiment of this aspect of the invention, the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In yet another embodiment, the cancer is non-small cell lung cancer (NSCLC). In yet another embodiment, the cancer is glioblastoma.

In yet another embodiment of this aspect of the invention, the compound or pharmaceutically acceptable salt thereof is administered as a pharmaceutical composition comprising the compound of the formula 1 and at least one pharmaceutically acceptable carrier. In another embodiment of this aspect, the compound of the formula 1 or a pharmaceutically acceptable salt thereof is administered as a pharmaceutical composition comprising the compound of formula 1 or a pharmaceutically acceptable salt thereof and at least one pharmaceutically acceptable carrier.

In another aspect the present invention provides a method of treating abnormal cell growth in a mammal in need of such treatment comprising administering to said mammal a therapeutically effective amount of a ROS kinase inhibitor. In one embodiment of this aspect of the invention, the abnormal cell growth is mediated by at least one genetically altered ROS kinase. In another embodiment, the abnormal cell growth is mediated by a fusion gene of ROS kinase. In another embodiment, the abnormal cell growth is mediated by a genetic deletion involving ROS kinase. In another embodiment, the fusion gene is SLC34A2-ROS or CD74-ROS. In another embodiment, the genetic deletion is FIG-ROS. In another embodiment, the abnormal cell growth is mediated by a fusion protein of ROS kinase. In another embodiment, the abnormal cell growth is mediated by a deletion protein involving ROS kinase. In another embodiment, the fusion protein is CD74-ROS. In another embodiment, the fusion protein is SLC34A2-ROS. In another embodiment, the deletion protein is FIG-ROS.

In another embodiment of this aspect, the ROS kinase inhibitor is a small molecule inhibitor of ROS kinase. In another embodiment, the ROS kinase inhibitor is an amino-pyridine compound or an amino-pyrazine compound. In another embodiment of each of the preceding aspects of the invention, the ROS kinase inhibitor is a compound of the formula 1:

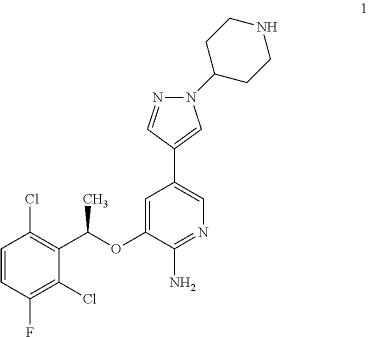 [SMILES: C[C@@H](Oc1cc(-c2cnn(C3CCNCC3)c2)cnc1N)c1c(Cl)ccc(F)c1Cl]

or a pharmaceutically acceptable salt thereof.

In another embodiment of this aspect of the invention, the abnormal cell growth is cancer. In another embodiment of each of the preceding aspects of the invention, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In yet another embodiment of this aspect of the invention, the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In yet another embodiment of each of the preceding aspects of the invention, the cancer is non-small cell lung cancer (NSCLC). In yet another embodiment, the cancer is glioblastoma.

In yet another embodiment of this aspect of the invention, the compound or pharmaceutically acceptable salt thereof is administered as a pharmaceutical composition comprising the compound of the formula 1 and at least one pharmaceutically acceptable carrier. In another embodiment of this aspect, the compound of the formula 1 or a pharmaceutically acceptable salt thereof is administered as a pharmaceutical composition comprising the compound of formula 1 or a pharmaceutically acceptable salt thereof and at least one pharmaceutically acceptable carrier.

In one embodiment of each of the preceding aspects of the invention, the mammal is a human. In another embodiment of each of the preceding aspects of the invention, the mammal is a dog.

In another aspect the present invention provides a method of treating cancer shown to be positive for at least one genetically altered ROS kinase in a mammal in need of such treatment comprising administering to said mammal a therapeutically effective amount of a ROS kinase inhibitor. In one embodiment of this aspect of the invention, the genetically altered ROS kinase is a fusion gene of ROS. In another embodiment, the fusion gene is SLC34A2-ROS or CD74-ROS. In another embodiment, the fusion gene is SLC34A2-ROS. In another embodiment, the fusion gene is CD74-ROS. In another embodiment, the genetically altered ROS kinase is a genetic deletion involving ROS kinase. In another embodiment, the genetic deletion is FIG-ROS. In another embodiment, the genetically altered ROS kinase is a fusion protein of ROS kinase. In another embodiment, the genetically altered ROS kinase is a deletion protein involving ROS kinase. In another embodiment, the fusion protein is SLC34A2-ROS or CD74-ROS. In another embodiment, the fusion protein is CD74-ROS. In another embodiment, the fusion protein is SLC34A2-ROS. In another embodiment, the deletion protein is FIG-ROS.

In another embodiment of this aspect, the ROS kinase inhibitor is a small molecule inhibitor of ROS kinase. In another embodiment, the ROS kinase inhibitor is an amino-pyridine compound or an amino-pyrazine compound. In another embodiment of each of the preceding aspects of the invention, the ROS kinase inhibitor is a compound of the formula 1:

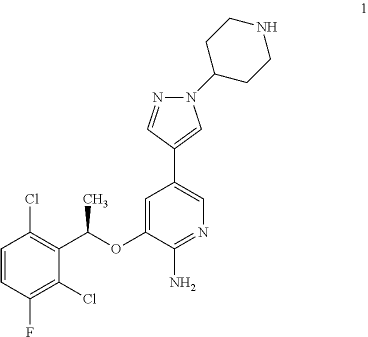 [SMILES: C[C@@H](Oc1cc(-c2cnn(C3CCNCC3)c2)cnc1N)c1c(Cl)ccc(F)c1Cl]

or a pharmaceutically acceptable salt thereof.

In another embodiment, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In yet another embodiment of this aspect of the invention, the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In yet another embodiment, the cancer is non-small cell lung cancer (NSCLC). In yet another embodiment, the cancer is glioblastoma.

In yet another embodiment of this aspect of the invention, the compound or pharmaceutically acceptable salt thereof is administered as a pharmaceutical composition comprising the compound of the formula 1 and at least one pharmaceutically acceptable carrier. In another embodiment of this aspect, the compound of the formula 1 or a pharmaceutically acceptable salt thereof is administered as a pharmaceutical composition comprising the compound of formula 1 or a pharmaceutically acceptable salt thereof and at least one pharmaceutically acceptable carrier.

In another aspect the present invention provides a method of treating ROS positive cancer comprising administering to a mammal in need of such treatment a therapeutically effective amount of a ROS kinase inhibitor. In one embodiment of this aspect of the invention, the ROS positive cancer is mediated by a fusion gene of ROS. In another embodiment, the fusion gene is SLC34A2-ROS or CD74-ROS. In another embodiment, the fusion gene is SLC34A2-ROS. In another embodiment, the fusion gene is CD74-ROS. In another embodiment, ROS positive cancer is mediated by a genetic deletion involving ROS kinase. In another embodiment, the genetic deletion is FIG-ROS. In another embodiment, the ROS positive cancer is mediated by a fusion protein of ROS kinase. In another embodiment, the ROS positive cancer is mediated by a deletion protein involving ROS kinase. In another embodiment, the fusion protein of ROS kinase is SLC34A2-ROS or CD74-ROS. In another embodiment, the fusion protein of ROS kinase is CD74-ROS. In another embodiment, the fusion protein of ROS kinase is SLC34A2-ROS. In another embodiment, the deletion protein of ROS kinase is FIG-ROS.

In another embodiment of this aspect, the ROS kinase inhibitor is a small molecule inhibitor of ROS kinase. In another embodiment, the ROS kinase inhibitor is an amino-pyridine compound or an amino-pyrazine compound. In another embodiment of each of the preceding aspects of the invention, the ROS kinase inhibitor is a compound of the formula 1:

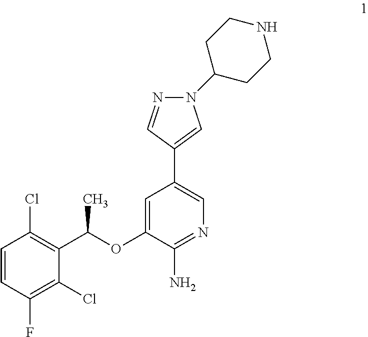 [SMILES: C[C@@H](Oc1cc(-c2cnn(C3CCNCC3)c2)cnc1N)c1c(Cl)ccc(F)c1Cl]

or a pharmaceutically acceptable salt thereof.

In another embodiment, the ROS positive cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In yet another embodiment of this aspect of the invention, the ROS positive cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In yet another embodiment, the ROS positive cancer is non-small cell lung cancer (NSCLC). In yet another embodiment, the ROS positive cancer is glioblastoma.

In some embodiments of this aspect, the compound of the formula 1 or a pharmaceutically acceptable salt thereof is administered as a pharmaceutical composition comprising the compound of formula 1 or a pharmaceutically acceptable salt thereof and at least one pharmaceutically acceptable carrier.

In another aspect the present invention provides a method comprising administering to a mammal having abnormal cell growth mediated by ROS kinase a therapeutically effective amount of a ROS kinase inhibitor. In one embodiment of this aspect of the invention, the abnormal cell growth is mediated by at least one genetically altered ROS kinase. In another embodiment, the abnormal cell growth is mediated by a fusion gene of ROS kinase. In another embodiment, the abnormal cell growth is mediated by a genetic deletion involving ROS kinase. In another embodiment, the fusion gene is SLC34A2-ROS or CD74-ROS. In another embodiment, the genetic deletion is FIG-ROS. In another embodiment, the abnormal cell growth is mediated by a fusion protein of ROS kinase. In another embodiment, the abnormal cell growth is mediated by a deletion protein involving ROS kinase. In another embodiment, the fusion protein is SLC34A2-ROS or CD74-ROS. In another embodiment, the deletion protein is FIG-ROS.

In another embodiment of this aspect, the ROS kinase inhibitor is a small molecule inhibitor of ROS kinase. In another embodiment, the ROS kinase inhibitor is an amino-pyridine compound or an amino-pyrazine compound. In another embodiment of each of the preceding aspects of the invention, the ROS kinase inhibitor is a compound of the formula 1:

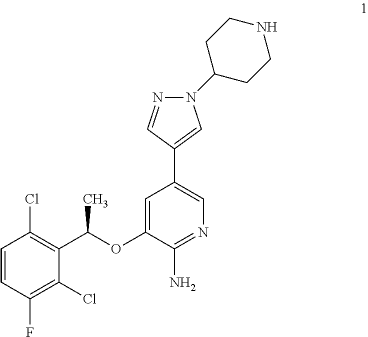 [SMILES: C[C@@H](Oc1cc(-c2cnn(C3CCNCC3)c2)cnc1N)c1c(Cl)ccc(F)c1Cl]

or a pharmaceutically acceptable salt thereof.

In another embodiment of this aspect of the invention, the abnormal cell growth is cancer. In another embodiment, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In yet another embodiment of this aspect of the invention, the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In yet another embodiment, the cancer is non-small cell lung cancer (NSCLC). In yet another embodiment, the cancer is glioblastoma.

In yet another embodiment of this aspect of the invention, the compound or pharmaceutically acceptable salt thereof is administered as a pharmaceutical composition comprising the compound of the formula 1 and at least one pharmaceutically acceptable carrier. In another embodiment of this aspect, the compound of the formula 1 or a pharmaceutically acceptable salt thereof is administered as a pharmaceutical composition comprising the compound of formula 1 or a pharmaceutically acceptable salt thereof and at least one pharmaceutically acceptable carrier.

In yet another embodiment of this aspect of the invention, the compound or pharmaceutically acceptable salt thereof is administered as a pharmaceutical composition comprising the compound of the formula 1 and at least one pharmaceutically acceptable carrier. In another embodiment of this aspect, the compound of the formula 1 or a pharmaceutically acceptable salt thereof is administered as a pharmaceutical composition comprising the compound of formula 1 or a pharmaceutically acceptable salt thereof and at least one pharmaceutically acceptable carrier.

In another aspect, the invention provides for a method comprising administering a therapeutically effective amount of a ROS kinase inhibitor to a patient that is known to be ROS positive. In one embodiment the patient has cancer that is mediated by at least one genetically altered ROS kinase. In another embodiment, the cancer is mediated by a fusion gene of ROS kinase. In another embodiment, the cancer is mediated by a genetic deletion involving ROS kinase. In another embodiment, the fusion gene is SLC34A2-ROS or CD74-ROS. In another embodiment, the genetic deletion is FIG-ROS. In another embodiment, the cancer is mediated by a fusion protein of ROS kinase. In another embodiment, the cancer is mediated by a deletion protein involving ROS kinase. In another embodiment, the fusion protein is SLC34A2-ROS or CD74-ROS. In another embodiment, the deletion protein is FIG-ROS.

In another embodiment of this aspect, the ROS kinase inhibitor is a small molecule inhibitor of ROS kinase. In another embodiment, the ROS kinase inhibitor is an amino-pyridine compound or an amino-pyrazine compound. In another embodiment of each of the preceding aspects of the invention, the ROS kinase inhibitor is a compound of the formula 1:

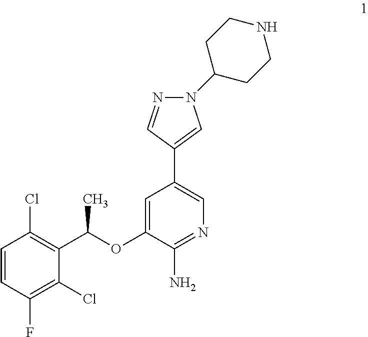 [SMILES: C[C@@H](Oc1cc(-c2cnn(C3CCNCC3)c2)cnc1N)c1c(Cl)ccc(F)c1Cl]

or a pharmaceutically acceptable salt thereof.

In another embodiment, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In yet another embodiment of this aspect of the invention, the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In yet another embodiment of each of the preceding aspects of the invention, the cancer is non-small cell lung cancer (NSCLC). In yet another embodiment, the cancer is glioblastoma.

In yet another embodiment of this aspect of the invention, the compound or pharmaceutically acceptable salt thereof is administered as a pharmaceutical composition comprising the compound of the formula 1 and at least one pharmaceutically acceptable carrier.

In yet another embodiment of this aspect of the invention, the compound or pharmaceutically acceptable salt thereof is administered as a pharmaceutical composition comprising the compound of the formula 1 and at least one pharmaceutically acceptable carrier. In another embodiment of this aspect, the compound of the formula 1 or a pharmaceutically acceptable salt thereof is administered as a pharmaceutical composition comprising the compound of formula 1 or a pharmaceutically acceptable salt thereof and at least one pharmaceutically acceptable carrier.

In another aspect, the present invention provides a method comprising,

i. identifying a patient having a cancer shown to be positive for at least one genetically altered ROS kinase; and

ii. administering to said patient a therapeutically effective amount of a ROS kinase inhibitor.

In one embodiment of this aspect of the invention, said genetically altered ROS kinase is a fusion gene of ROS. In another embodiment, the fusion gene is SLC34A2-ROS or CD74-ROS. In another embodiment, the fusion gene is SLC34A2-ROS. In another embodiment, the fusion gene is CD74-ROS. In another embodiment, said genetically altered ROS kinase is a genetic deletion involving ROS kinase. In another embodiment, the genetic deletion is FIG-ROS. In another embodiment, said genetically altered ROS kinase is a fusion protein of ROS kinase. In another embodiment, said genetically altered ROS kinase is a deletion protein involving ROS kinase. In another embodiment, the fusion protein is SLC34A2-ROS or CD74-ROS. In another embodiment, the fusion protein is SLC34A2-ROS. In another embodiment, the fusion protein is CD74-ROS. In another embodiment, the deletion protein is FIG-ROS. In some such embodiments of this aspect, the method comprises (i) identifying a patient having a cancer shown to be positive for at least one genetically altered ROS kinase; and (ii) administering to said patient a therapeutically effective amount of a ROS kinase inhibitor, thereby treating said cancer. In some embodiments of this aspect, said treating results in reversing or inhibiting the progression of cancer.

In another embodiment of this aspect, the ROS kinase inhibitor is a small molecule inhibitor of ROS kinase. In another embodiment, the ROS kinase inhibitor is an amino-pyridine compound or an amino-pyrazine compound. In another embodiment of each of the preceding aspects of the invention, the ROS kinase inhibitor is a compound of the formula 1:

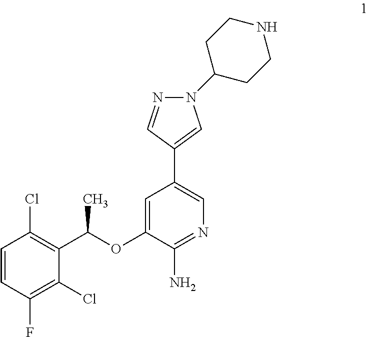 [SMILES: C[C@@H](Oc1cc(-c2cnn(C3CCNCC3)c2)cnc1N)c1c(Cl)ccc(F)c1Cl]

or a pharmaceutically acceptable salt thereof.

In another embodiment of this aspect of the invention, the abnormal cell growth is cancer. In another embodiment of each of the preceding aspects of the invention, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In yet another embodiment of this aspect of the invention, the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In yet another embodiment of each of the preceding aspects of the invention, the cancer is non-small cell lung cancer (NSCLC). In yet another embodiment, the cancer is glioblastoma.

In yet another embodiment of this aspect of the invention, the compound or pharmaceutically acceptable salt thereof is administered as a pharmaceutical composition comprising the compound of the formula 1 and at least one pharmaceutically acceptable carrier. In another embodiment of this aspect, the compound of the formula 1 or a pharmaceutically acceptable salt thereof is administered as a pharmaceutical composition comprising the compound of formula 1 or a pharmaceutically acceptable salt thereof and at least one pharmaceutically acceptable carrier.

In one aspect the present invention provides a use of a ROS kinase inhibitor for the preparation of a medicament useful in the treatment of cancer in a human in need of such treatment comprising, administering to said mammal a therapeutically effective amount of a ROS kinase inhibitor of the formula 1

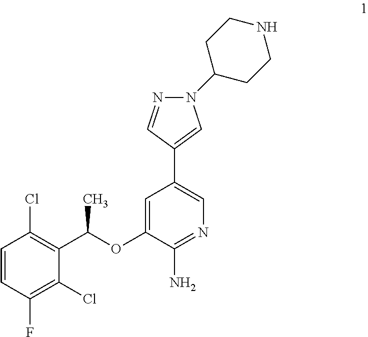 [SMILES: C[C@@H](Oc1cc(-c2cnn(C3CCNCC3)c2)cnc1N)c1c(Cl)ccc(F)c1Cl]

or a pharmaceutically acceptable salt thereof, wherein the cancer is mediated by at least one genetically altered ROS. In one embodiment of this aspect of the invention, the cancer is mediated by a fusion gene of ROS. In another embodiment of this aspect, the fusion gene of ROS is SLC34A2-ROS gene or CD74-ROS gene. In another embodiment of this aspect, the cancer is mediated by a genetic deletion involving ROS kinase. In another embodiment of this aspect, the genetic deletion is FIG-ROS gene. In another embodiment of this aspect, the cancer is mediated by a genetically altered ROS kinase. In another embodiment of this aspect, the genetically altered ROS kinase is a ROS fusion. In another embodiment of this aspect, the ROS fusion is SLC34A2-ROS kinase or CD74-ROS kinase. In another embodiment of this aspect, the cancer is mediated by a deletion protein involving ROS kinase. In another embodiment of this aspect, the deletion protein is FIG-ROS kinase.

In another embodiment of this aspect, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof. In another embodiment of this aspect, the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In another embodiment of this aspect, the cancer is non-small cell lung cancer (NSCLC). In another embodiment of this aspect, the cancer is glioblastoma. In another embodiment of this aspect, the compound of the formula 1 is administered as a pharmaceutical composition comprising the compound of the formula 1 and at least one pharmaceutically acceptable carrier.

In another aspect, the present invention provides a use of a ROS kinase inhibitor for the preparation of a medicament useful in the treatment of a cancer mediated by at least one genetically altered ROS kinase. In one embodiment, ROS kinase inhibitor is a small molecule inhibitor of ROS kinase. In another embodiment, the ROS kinase inhibitor is an amino-pyridine compound or an amino-pyrazine compound. In another embodiment, the ROS kinase inhibitor is a compound of the formula 1:

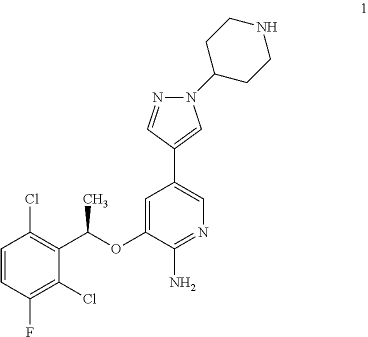 [SMILES: C[C@@H](Oc1cc(-c2cnn(C3CCNCC3)c2)cnc1N)c1c(Cl)ccc(F)c1Cl]

or a pharmaceutically acceptable salt thereof. In one embodiment of this aspect of the invention, said genetically altered ROS kinase is a fusion gene of ROS. In another embodiment, the fusion gene is SLC34A2-ROS or CD74-ROS. In another embodiment, the fusion gene is SLC34A2-ROS. In another embodiment, the fusion gene is CD74-ROS. In another embodiment, said genetically altered ROS kinase is a genetic deletion involving ROS kinase. In another embodiment, the genetic deletion is FIG-ROS. In another embodiment, said genetically altered ROS kinase is a fusion protein of ROS kinase. In another embodiment, said genetically altered ROS kinase is a deletion protein involving ROS kinase. In another embodiment, the fusion protein is SLC34A2-ROS or CD74-ROS. In another embodiment, the fusion protein is SLC34A2-ROS. In another embodiment, the fusion protein is CD74-ROS. In another embodiment, the deletion protein is FIG-ROS. In yet another embodiment of this aspect of the invention, the ROS positive cancer is non-small cell lung cancer (NSCLC). In yet another embodiment, the ROS positive cancer is glioblastoma.

In another aspect, the present invention provides a use of a ROS kinase inhibitor for the preparation of a medicament useful in the treatment of a ROS positive cancer. In one embodiment, ROS kinase inhibitor is a small molecule inhibitor of ROS kinase. In another embodiment, the ROS kinase inhibitor is an amino-pyridine compound or an amino-pyrazine compound. In another embodiment, the ROS kinase inhibitor is a compound of the formula 1:

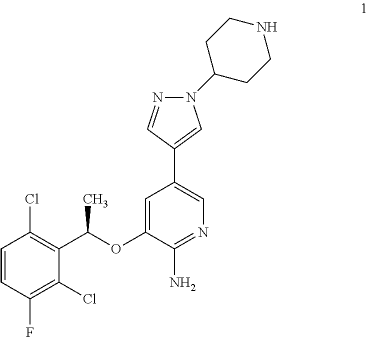 [SMILES: C[C@@H](Oc1cc(-c2cnn(C3CCNCC3)c2)cnc1N)c1c(Cl)ccc(F)c1Cl]

or a pharmaceutically acceptable salt thereof. In one embodiment of this aspect of the invention, said genetically altered ROS kinase is a fusion gene of ROS. In another embodiment, the fusion gene is SLC34A2-ROS or CD74-ROS. In another embodiment, the fusion gene is SLC34A2-ROS. In another embodiment, the fusion gene is CD74-ROS. In another embodiment, said genetically altered ROS kinase is a genetic deletion involving ROS kinase. In another embodiment, the genetic deletion is FIG-ROS. In another embodiment, said genetically altered ROS kinase is a fusion protein of ROS kinase. In another embodiment, said genetically altered ROS kinase is a deletion protein involving ROS kinase. In another embodiment, the fusion protein is SLC34A2-ROS or CD74-ROS. In another embodiment, the fusion protein is SLC34A2-ROS. In another embodiment, the fusion protein is CD74-ROS. In another embodiment, the deletion protein is FIG-ROS. In yet another embodiment of this aspect of the invention, the ROS positive cancer is non-small cell lung cancer (NSCLC). In yet another embodiment, the ROS positive cancer is glioblastoma.

In another aspect, the present invention provides a kit comprising a pharmaceutical composition of a ROS kinase inhibitor and a set of instructions for administering said pharmaceutical composition to a patient having a ROS positive cancer. In one embodiment of this aspect of the invention, the ROS positive cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In yet another embodiment of this aspect of the invention, the ROS positive cancer is non-small cell lung cancer (NSCLC). In yet another embodiment, the ROS positive cancer is glioblastoma.

In another aspect, the present invention provides a kit comprising a pharmaceutical composition of a ROS kinase inhibitor and a set of instructions for administering said pharmaceutical composition to a patient having a ROS positive cancer. In one embodiment, said ROS positive cancer is mediated by at least one genetically altered ROS kinase. In another embodiment, said genetically altered ROS kinase is a fusion gene of ROS. In another embodiment, the fusion gene is SLC34A2-ROS or CD74-ROS. In another embodiment, the fusion gene is SLC34A2-ROS. In another embodiment, the fusion gene is CD74-ROS. In another embodiment, said genetically altered ROS kinase is a genetic deletion involving ROS kinase. In another embodiment, the genetic deletion is FIG-ROS. In another embodiment, said genetically altered ROS kinase is a fusion protein of ROS kinase. In another embodiment, said genetically altered ROS kinase is a deletion protein involving ROS kinase. In another embodiment, the fusion protein is SLC34A2-ROS or CD74-ROS. In another embodiment, the fusion protein is SLC34A2-ROS. In another embodiment, the fusion protein is CD74-ROS. In another embodiment, the deletion protein is FIG-ROS. In one embodiment of this aspect of the invention, the ROS positive cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In yet another embodiment of this aspect of the invention, the ROS positive cancer is non-small cell lung cancer (NSCLC). In yet another embodiment, the ROS positive cancer is glioblastoma.

In another aspect, the present invention provides a kit comprising a pharmaceutical composition of crizotinib and a set of instructions for administering said pharmaceutical composition to a patient having a ROS positive cancer. In one embodiment, said ROS positive cancer is mediated by at least one genetically altered ROS kinase. In another embodiment, said genetically altered ROS kinase is a fusion gene of ROS. In another embodiment, the fusion gene is SLC34A2-ROS or CD74-ROS. In another embodiment, the fusion gene is SLC34A2-ROS. In another embodiment, the fusion gene is CD74-ROS. In another embodiment, said genetically altered ROS kinase is a genetic deletion involving ROS kinase. In another embodiment, the genetic deletion is FIG-ROS. In another embodiment, said genetically altered ROS kinase is a fusion protein of ROS kinase. In another embodiment, said genetically altered ROS kinase is a deletion protein involving ROS kinase. In another embodiment, the fusion protein is SLC34A2-ROS or CD74-ROS. In another embodiment, the fusion protein is SLC34A2-ROS. In another embodiment, the fusion protein is CD74-ROS. In one embodiment of this aspect of the invention, the ROS positive cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In yet another embodiment of this aspect of the invention, the ROS positive cancer is non-small cell lung cancer (NSCLC). In yet another embodiment, the ROS positive cancer is glioblastoma.

In yet another aspect, the present invention provides a method of inhibiting ROS kinase activity in a cell by administering a compound of the formula 1:

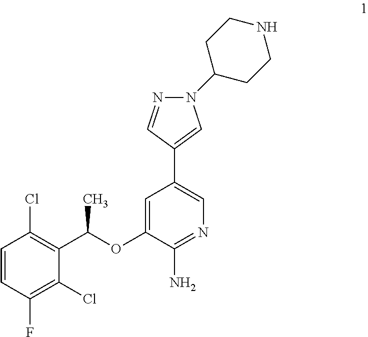 [SMILES: C[C@@H](Oc1cc(-c2cnn(C3CCNCC3)c2)cnc1N)c1c(Cl)ccc(F)c1Cl]

or a pharmaceutically acceptable salt thereof.

In another aspect, the invention provides a method of treating cancer in a mammal comprising administering to said mammal a therapeutically effective amount of 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof, wherein the cancer is mediated by at least one genetically altered ROS. In some such embodiments, the at least one genetically altered ROS is a genetically altered ROS gene or a genetically altered ROS protein.

In some embodiments of this aspect, said treating results in reversing or inhibiting the progression of cancer. In frequent embodiments of this aspect, the mammal is a human.

In frequent embodiments of this aspect, the at least one genetically altered ROS is a genetically altered ROS gene, such as a ROS fusion gene. In some such embodiments, the ROS fusion gene is the SLC34A2-ROS gene or the CD74-ROS gene. In other such embodiments, the ROS fusion gene is the FIG-ROS gene.

In frequent embodiments of this aspect, the at least one genetically altered ROS is a genetically altered ROS protein, such as a ROS fusion protein. In some such embodiments, the ROS fusion protein is the SLC34A2-ROS kinase or the CD74-ROS kinase. In other such embodiments, the ROS fusion protein is the FIG-ROS kinase.

In some embodiments of this aspect, the invention provides a method of reversing or inhibiting the progression of cancer in a mammal comprising administering to said mammal a therapeutically effective amount of 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof, wherein the cancer is mediated by a ROS fusion gene. In some such embodiments, the ROS fusion gene is the SLC34A2-ROS gene. In other such embodiments, the ROS fusion gene is CD74-ROS gene. In still other such embodiments, the ROS fusion gene is the FIG-ROS gene. In certain embodiments, the ROS fusion gene is selected from the group consisting of the SLC34A2-ROS gene, the CD74-ROS gene and the FIG-ROS gene.

In other embodiments of this aspect, the invention provides a method of reversing or inhibiting the progression of cancer in a mammal comprising administering to said mammal a therapeutically effective amount of 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof, wherein the cancer is mediated by a ROS fusion protein. In some such embodiments, the ROS fusion protein is the SLC34A2-ROS kinase. In other such embodiments, the ROS fusion protein is the CD74-ROS kinase. In still other such embodiments, the ROS fusion protein is the FIG-ROS kinase. In certain embodiments, the ROS fusion protein is selected from the group consisting of the SLC34A2-ROS kinase, the CD74-ROS kinase and the FIG-ROS kinase.

In some embodiments of this aspect, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In other embodiments of this aspect, the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In some embodiments of this aspect, the cancer is non-small cell lung cancer (NSCLC). In other embodiments of this aspect, the cancer is glioblastoma.

In frequent embodiments of this aspect, 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof is administered as a pharmaceutical composition comprising 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof and at least one pharmaceutically acceptable carrier.

In some embodiments of this aspect, the method further comprises a step of identifying a mammal having a cancer characterized by at least one genetically altered ROS, such as a genetically altered ROS gene or a genetically altered ROS protein, prior to said administering step. In some such embodiments, the cancer is characterized as having a genetically altered ROS polynucleotide and/or a genetically altered ROS polypeptide.

In yet another aspect, the invention provides a method of treating cancer in a mammal comprising: (i) identifying a mammal having a cancer characterized by at least one genetically altered ROS; and (ii) administering to said mammal a therapeutically effective amount of 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof. In some such embodiments, the at least one genetically altered ROS is a genetically altered ROS gene or a genetically altered ROS protein.

In some embodiments of this aspect, said treating results in reversing or inhibiting the progression of cancer. In frequent embodiments of this aspect, the mammal is a human.

In some embodiments of this aspect, the at least one genetically altered ROS is a genetically altered ROS gene, for example a ROS fusion gene. In some such embodiments, the ROS fusion gene is the SLC34A2-ROS gene or the CD74-ROS gene. In other such embodiments, the ROS fusion gene is the FIG-ROS gene.

In some embodiments of this aspect, the at least one genetically altered ROS is a genetically altered ROS protein, for example a ROS fusion protein. In some such embodiments, the ROS fusion protein is the SLC34A2-ROS kinase or the CD74-ROS kinase. In other such embodiments, the ROS fusion protein is the FIG-ROS kinase.

In some embodiments of this aspect, the invention provides a method of reversing or inhibiting the progression of cancer in a mammal comprising (i) identifying a mammal having a cancer characterized by at least one ROS fusion gene; and (ii) administering to said mammal a therapeutically effective amount of 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof. In some such embodiments, the ROS fusion gene is the SLC34A2-ROS gene. In other such embodiments, the ROS fusion gene is CD74-ROS gene. In still other such embodiments, the ROS fusion gene is the FIG-ROS gene. In certain embodiments, the ROS fusion gene is selected from the group consisting of the SLC34A2-ROS gene, the CD74-ROS gene and the FIG-ROS gene.

In some embodiments of this aspect, the invention provides a method of reversing or inhibiting the progression of cancer in a mammal comprising (i) identifying a mammal having a cancer characterized by at least one ROS fusion protein; and (ii) administering to said mammal a therapeutically effective amount of 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof. In some such embodiments, the ROS fusion protein is the SLC34A2-ROS kinase. In other such embodiments, the ROS fusion protein is the CD74-ROS kinase. In still other such embodiments, the ROS fusion protein is the FIG-ROS kinase. In certain embodiments, the ROS fusion protein is selected from the group consisting of the SLC34A2-ROS kinase, the CD74-ROS kinase and the FIG-ROS kinase.

In some embodiments of this aspect, the cancer is characterized as having a genetically altered ROS polynucleotide and/or a genetically altered ROS polypeptide.

In some embodiments of this aspect, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In other embodiments of this aspect, the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer. In some embodiments of this aspect, the cancer is non-small cell lung cancer (NSCLC). In other embodiments of this aspect, the cancer is glioblastoma.

In frequent embodiments of this aspect, 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof is administered as a pharmaceutical composition comprising 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof and at least one pharmaceutically acceptable carrier.

### BRIEF DESCRIPTION OF THE DRAWINGS

FIG. 1: Concentration dependent inhibition of SLC34A2-ROS phosphorylation in U138MG cells and HCC78 cells by crizotinib.

FIG. 2: Concentration dependent inhibition of HCC78 cell viability by crizotinib.

FIG. 3: Concentration dependent inhibition of SLC34A2-ROS phosphorylation and ROS mediated signal transduction by crizotinib in HCC78 human NSCLC cells.

FIG. 4: Dose-dependent increase by crizotinib in cleaved Caspase3 levels in HCC78 human NSCLC cells that harbor SLC34A2-ROS fusion.

FIG. 5: Cytoreductive effects of crizotinib in a panel of ROS fusion engineered 3T3-ROS tumor models that harbor human CD74-ROS, SLC34A2-ROS (L), SLC34A2-ROS (S), FIG-ROS (L) and FIG-ROS (S) in nude mice.

FIG. 6: Dose-dependent inhibition by crizotinib of ROS phosphorylation (A) and tumor growth (B) in 3T3-CD74-ROS xenograft model in Nude mice.

FIG. 7: Dose-dependent inhibition of tumor growth by crizotinib in the 3T3-SLC34A2-ROS(L) xenograft model in Nude mice.

### DETAILED DESCRIPTION OF THE INVENTION

Unless indicated otherwise, all references herein to the inventive compounds include references to salts, solvates, hydrates and complexes thereof, and to solvates, hydrates and complexes of salts thereof, including polymorphs, stereoisomers, and isotopically labeled versions thereof.

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice or testing of the present invention, preferred methods and materials are described herein. In describing the embodiments and claiming the invention, certain terminology will be used in accordance with the definitions set out below.

As used herein, the singular forms “a,” “an,” and “the” include plural references unless the context clearly dictates otherwise. Thus, e.g., references to “the method” includes one or more methods, and/or steps of the type described herein and/or which will become apparent to one of ordinary skill in the art upon reading this disclosure.

As used herein, unless otherwise indicated, the term “abnormal cell growth” refers to cell growth that is independent of normal regulatory mechanisms (e.g., loss of contact inhibition).

As used herein, unless otherwise indicated, the term, the term “administering” refers to the act of self-administering wherein a patient ingests a therapeutic as described herein by their own effort, the act of administering wherein a patient ingests a therapeutic as described herein through the effort of another (e.g., a doctor, a nurse, a family member, or an IV). Administering also includes the act of prescribing a therapeutic as described herein. The term “administration”, as used herein, unless otherwise indicated, refers to the act of treating as “administering” is defined immediately above.

As used herein, “antibody” or “antibodies” refer to all types of immunoglobulins, including IgG, IgM, IgA, IgD, and IgE, including Fab or antigen-recognition fragments thereof, including chimeric, polyclonal, and monoclonal antibodies. The term “humanized antibody”, as used herein, refers to antibody molecules in which amino acids have been replaced in the nonantigen binding regions in order to more closely resemble a human antibody, while still retaining the original binding ability.

The term “biological sample” is used herein in its broadest sense, and means any biological sample suspected of containing SLC34A2-ROS fusion, CD74-ROS fusion, FIG-ROS fusion or truncated ROS polynucleotides or polypeptides or fragments thereof, and may comprise a cell, chromosomes isolated from a cell (e.g., a spread of metaphase chromosomes), genomic DNA (in solution or bound to a solid support such as for Southern analysis), RNA (in solution or bound to a solid support such as for northern analysis), cDNA (in solution or bound to a solid support), an extract from cells, blood, urine, marrow, or a tissue, and the like.

As used herein, the term “deletion gene” refers to a gene that results from a genetic event whereby two genes from different locations on the same chromosome in the genome become fused through a deletion of nucleotides in between the two genes (also referred to as a “genetic deletion”). Deletion genes include but are not limited to the FIG-ROS gene described above.

As used herein, the term “fusion gene” refers to a gene that results from a genetic event whereby two genes from different locations in the genome become fused, translocated, or inverted to create a new gene. Specific examples of fusion genes include but are not limited to the fusion of the SLC34A2 gene and the ROS gene to form the SLC34A2-ROS gene, and the fusion of the CD74 gene and the ROS gene to form the CD74-ROS gene.

As used herein, the term “genetically altered ROS” refers to any of the ROS fusions or deletions described herein, whether genomic DNA, nucleotides, proteins or polypeptides. The term “genetically altered ROS polynucleotide” refers to the polynucleotide encoding any of the genetically altered ROS proteins described herein. The term “genetically altered ROS protein” refers to any of the fusion, deletion, truncations or mutations described herein. The term “genetically altered ROS protein” as used herein is used interchangeably with “genetically altered ROS polypeptide”. Preferred genetically altered ROS proteins include “ROS fusions”. Preferred ROS fusions include but are not limited to SLC34A2-ROS fusion protein and CD74-ROS fusion protein. Preferred genetically altered ROS polypeptides include SLC34A2-ROS fusion polypeptides and CD74-ROS fusion polypeptides.

As used herein, “ROS kinase” refers to any protein described herein that contains the kinase portion of the ROS protein. ROS kinase includes but is not limited to the genetically altered ROS proteins described herein and to the wild-type ROS protein. The term “genetically altered ROS kinase” refers to the protein or polypeptide encoded by a genetically altered ROS polynucleotide.

As used herein, the term “ROS polypeptide-specific reagent” refers to any reagent that is specific for any of the ROS kinases described herein, such as antibodies, AQUA peptides, nucleic acid probes, nucleic acid primers, and the like. For example, a preferred “ROS polypeptide-specific reagent” is an antibody specific for any of the genetically altered ROS kinases described herein. More preferably, as used herein a “ROS polypeptide-specific reagent” is an antibody specific for a SLC34A2-ROS fusion polypeptide and/or a CD74-ROS fusion polypeptide and/or a FIG-ROS fusion polypeptide. When the “ROS polypeptide-specific reagent” is an antibody, the reagent may be referred to herein as a “ROS polypeptide-specific antibody”. Such a ROS polypeptide-specific antibody is for example, a “SLC34A2-ROS fusion polypeptide antibody”, a “SLC34A2-ROS fusion protein antibody” or a “FIG-ROS fusion protein antibody”.

As used herein, unless otherwise indicated, the term “treating”, means reversing, alleviating, inhibiting the progress of the disorder or condition to which such term applies, or one or more symptoms of such disorder or condition. The term “treatment”, as used herein, unless otherwise indicated, refers to the act of treating as “treating” is defined immediately above. The term “treatment” includes “administering” or “administration” as described above.

As used herein the term “pharmaceutically acceptable salts” includes acid addition and base salts (including disalts).

Suitable acid addition salts are formed from acids which form non-toxic salts. Examples include the acetate, aspartate, benzoate, besylate, bicarbonate/carbonate, bisulphate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

For a review on suitable pharmaceutically acceptable salts, see “Handbook of Pharmaceutical Salts: Properties, Selection, and Use” by Stahl and Wermuth (Wiley-VCH, Weinheim, Germany, 2002), the disclosure of which is incorporated herein by reference in its entirety.

A pharmaceutically acceptable salt of the inventive compounds can be readily prepared by mixing together solutions of the compound and the desired acid or base, as appropriate. The salt may precipitate from solution and be collected by filtration or may be recovered by evaporation of the solvent. The degree of ionization in the salt may vary from completely ionized to almost non-ionized.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when the solvent is water. Pharmaceutically acceptable solvates in accordance with the invention include hydrates and solvates wherein the solvent of crystallization may be isotopically substituted, e.g. D₂O, d₆-acetone, d₆-DMSO.

The invention also includes isotopically-labeled compounds, which are identical to the compound of the formula 1, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention and pharmaceutically acceptable salts of said compounds, which contain the aforementioned isotopes and/or other isotopes of other atoms, are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. An isotopically labeled compound of the formula 1 of this invention can generally be prepared by carrying out the procedures described for the non-labeled compound, substituting a readily available isotopically labeled reagent for a non-isotopically labeled reagent.

Also included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see J Pharm Sci, 64 (8), 1269-1288 by Haleblian (August 1975), the disclosure of which is incorporated herein by reference in its entirety

Diagnostic Testing

A number of assay formats known to those skilled in the art may be used in connection with the present invention as diagnostic tests to determine the presence or absence of a genetically altered ROS in a biological sample. When a diagnostic test returns a test result showing that a biological sample contains a genetically altered ROS, the patient from which the biological sample was taken is considered ROS positive. Similarly, when a diagnostic test returns a test result showing that a biological sample, where the biological sample is a cancer tumor biopsy, contains a genetically altered ROS, the cancer is considered a ROS positive cancer. In particular, where the biological sample comprises cancer cells, the cancer can be characterized as containing a genetically altered ROS gene or a genetically altered ROS protein, such as a ROS fusion gene or ROS fusion protein, by detecting the presence of a genetically altered ROS polynucleotide and/or polypeptide using techniques known to those of skill in the art or as described herein.

Immunoassays

Immunoassays useful in the practice of the methods of the invention may be homogenous immunoassays or heterogeneous immunoassays. In a homogeneous assay the immunological reaction usually involves a mutant ROS polypeptide-specific reagent (e.g. a SLC34A2-ROS fusion polypeptide-specific antibody, a CD74-ROS fusion polypeptide-specific antibody or a FIG-ROS fusion polypeptide-specific antibody), a labeled analyte, and the biological sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radio-isotopes, fluorescent dyes, enzymes, bacteriophages, coenzymes, and so forth. Semi-conductor nanocrystal labels, or “quantum dots”, may also be advantageously employed, and their preparation and use has been well described (See generally, K. Barovsky, Nanotech. Law & Bus. 1 (2): Article 14 (2004) and patents cited therein).

In a heterogeneous assay approach, the reagents are usually the biological sample, a mutant ROS kinase polypeptide-specific reagent (e.g., an antibody), and suitable means for producing a detectable signal. Biological samples as further described below may be used. The antibody is generally immobilized on a support, such as a bead, plate or slide, and contacted with the sample suspected of containing the antigen in a liquid phase. The support is then separated from the liquid phase and either the support phase or the liquid phase is examined for a detectable signal employing means for producing such signal. The signal is related to the presence of the analyte in the biological sample. Means for producing a detectable signal include the use of radioactive labels, fluorescent labels, enzyme labels, quantum dots, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof, which may be useful for carrying out the methods disclosed herein, are well known in the art (See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”)). Conditions suitable for the formation of reagent-antibody complexes are well known to those of skill in the art. The concentration of detectable reagent should be sufficient such that the binding of SLC34A2-ROS fusion polypeptide is detectable compared to background.

Antibodies useful in the practice of the methods disclosed herein (e.g., IHC, Western blot, immune-fluorescence, and flow cytometry) include, without limitation, antibodies that specifically bind to either full length SLC34A2 or CD74 (e.g., bind to the N-terminus of the protein) or to full length ROS (e.g., bind an epitope in the kinase domain of ROS). Such antibodies may be commercially available (see, e.g., the ROS-specific polyclonal antibody sold by Abcam, Inc., Cambridge Mass. as Product ab5512). Where the antibody used specifically binds to full-length ROS or full-length SLC34A2, such in a Western blotting analysis or by flow cytometry, an additional method to detect the presence of a mutant ROS polypeptide or polynucleotide of the invention (e.g., an SLC34A2-ROS polypeptide or polynucleotide) may be employed on the same sample. For example, flow cytometry on permeabilized cells may be performed with the Abcam's ab5512 antibody, followed by lysis of the cells and PCR analysis of the genetic material (e.g., mRNA or genomic DNA) using PCR primer specific for (i.e., that hybridize to) the 5′ end of a cDNA encoding SLC34A2 or CD74 (e.g., the forward primer) and to the complement of the 3′ end of a cDNA encoding ROS (e.g., the reverse primer).

All antibodies for use in the methods of the invention may be conjugated to a solid support suitable for a diagnostic assay (e.g., beads, plates, slides or wells formed from materials such as latex or polystyrene) in accordance with known techniques, such as precipitation. Antibodies or other ROS polypeptide-specific reagents may likewise be conjugated to detectable groups such as radiolabels (e.g., 35S, 1251, 131 1), enzyme labels (e.g., horseradish peroxidase, alkaline phosphatase), and fluorescent labels (e.g., fluorescein) in accordance with known techniques.

Cell-based assays, such flow cytometry (FC), immunohistochemistry (IHC), or immunofluorescence (IF) are particularly desirable in practicing the methods of the invention, since such assay formats are clinically-suitable, allow the detection of genetically altered ROS protein expression in vivo, and avoid the risk of artifact changes in activity resulting from manipulating cells obtained from, e.g. a tumor sample in order to obtain extracts. Accordingly, in some preferred embodiment, the methods of the invention are implemented in a flow cytometry (FC), immuno-histochemistry (IHC), or immunofluorescence (IF) assay format.

Flow cytometry (FC) may be employed to determine the expression of genetically altered ROS protein in a mammalian tumor before, during, and after treatment with a drug targeted at inhibiting ROS kinase activity. For example, tumor cells from a fine needle aspirate may be analyzed by flow cytometry for SLC34A2-ROS fusion polypeptide expression or CD74-ROS fusion polypeptide expression and/or activation, as well as for markers identifying cancer cell types, etc., if so desired. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 2% paraformaldehyde for 10 minutes at 37° C. followed by permeabilization in 90% methanol for 30 minutes on ice. Cells may then be stained with the primary ROS polypeptide-specific antibody, washed and labeled with a fluorescent-labeled secondary antibody. The cells would then be analyzed on a flow cytometer (e.g. a Beckman Coulter FC500) according to the specific protocols of the instrument used. Such an analysis would identify the level of expressed SLC34A2-ROS fusion polypeptide or CD74-ROS fusion polypeptide in the tumor. Similar analysis after treatment of the tumor with a ROS-inhibiting therapeutic would reveal the responsiveness of a SLC34A2-ROS fusion polypeptide-expressing tumor or the CD74-ROS fusion polypeptide-expressing tumor to the targeted inhibitor of ROS kinase.

Immunohistochemical (IHC) staining may be also employed to determine the expression and/or activation status of genetically altered ROS protein in a mammalian cancer (e.g. NSCLC) before, during, and after treatment with a drug targeted at inhibiting ROS kinase activity. IHC may be carried out according to well-known techniques. (See for example, ANTIBODIES: A LABORATORY MANUAL, Chapter 10, Harlow & Lane Eds., Cold Spring Harbor Laboratory (1988)). Briefly, and by way of example, paraffin-embedded tissue (e.g. tumor tissue from a biopsy) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary anti-SLC34A2-ROS fusion polypeptide antibody or anti-CD74-ROS fusion polypeptide antibody and secondary antibody; and finally detecting using ABC avidin/biotin method according to manufacturer's instructions. Immunofluorescence (IF) assays may be also employed to determine the expression and/or activation status of SLC34A2-ROS fusion polypeptide or CD74-ROS fusion polypeptide in a mammalian cancer before, during, and after treatment with a drug targeted at inhibiting ROS kinase activity. IF may be carried out according to well-known techniques. See, e.g., J. M. Polak and S. Van Noorden (1997) INTRODUCTION TO IMMUNOCYTOCHEMISTRY, 2^{nd} Ed.; ROYAL MICROSCOPY SOCIETY MICROSCOPY HANDBOOK 37, BioScientific/Springer-Verlag. Briefly, and by way of example, patient samples may be fixed in paraformaldehyde followed by methanol, blocked with a blocking solution such as horse serum, incubated with the primary antibody against SLC34A2-ROS fusion polypeptide, CD74-ROS fusion polypeptide or FIG-ROS fusion polypeptide followed by a secondary antibody labeled with a fluorescent dye such as Alexa 488 and analyzed with an epifluorescent microscope.

Antibodies employed in the above-described assays may be advantageously conjugated to fluorescent dyes (e.g. Alexa488, PE), or other labels, such as quantum dots, for use in multi-parametric analyses along with other signal transduction (EGFR, phospho-AKT, phospho-Erk 1/2) and/or cell marker (cytokeratin) antibodies. A variety of other protocols, including enzyme-linked immunosorbent assay (ELISA), radio-immunoassay (RIA), and fluorescent-activated cell sorting (FACS), for measuring genetically altered ROS polypeptides are known in the art and provide a basis for diagnosing altered or abnormal levels of SLC34A2-ROS fusion polypeptide, CD74-ROS fusion polypeptide or FIG-ROS fusion polypeptide expression. Normal or standard values for SLC34A2-ROS fusion polypeptide, CD74-ROS fusion polypeptide or FIG-ROS fusion polypeptide expression are established by combining body fluids or cell extracts taken from normal mammalian subjects, preferably human, with antibody to SLC34A2-ROS fusion polypeptide, CD74-ROS fusion polypeptide or FIG-ROS fusion polypeptide under conditions suitable for complex formation. The amount of standard complex formation may be quantified by various methods, but preferably by photometric means. Quantities of SLC34A2-ROS fusion polypeptide, CD74-ROS fusion polypeptide or FIG-ROS fusion polypeptide expressed in subject, control, and disease samples from biopsied tissues are compared with the standard values. Deviation between standard and subject values establishes the parameters for diagnosing disease.

Peptide & Nucleotide Assays

Similarly, AQUA peptides for the detection/quantification of expressed genetically altered ROS polypeptide in a biological sample comprising cells from a tumor may be prepared and used in standard AQUA assays, as described in detail in Section E above. Accordingly, in some preferred embodiments of the methods of the invention, the ROS polypeptide-specific reagent comprises a heavy isotope labeled phosphopeptide (AQUA peptide) corresponding to a peptide sequence comprising the fusion junction of SLC34A2-ROS fusion polypeptide, CD74-ROS fusion polypeptide or FIG-ROS fusion polypeptide. ROS polypeptide-specific reagent useful in practicing the methods of the invention may also be mRNA, oligonucleotide or DNA probes that can directly hybridize to, and detect, fusion or truncated polypeptide expression transcripts in a biological sample.

Briefly, and by way of example, formalin-fixed, paraffin-embedded patient samples may be probed with a fluorescein-labeled RNA probe followed by washes with formamide, SSC and PBS and analysis with a fluorescent microscope. Polynucleotides encoding genetically altered ROS polypeptide may also be used for diagnostic purposes. The polynucleotides that may be used include oligonucleotide sequences, antisense RNA and DNA molecules, and PNAs. The polynucleotides may be used to detect and quantitate gene expression in biopsied tissues in which expression of SLC34A2-ROS fusion polypeptide, CD74-ROS fusion polypeptide or deletion ROS polypeptide may be correlated with disease. The diagnostic assay may be used to distinguish between absence, presence, and excess expression of SLC34A2-ROS fusion polypeptide, CD74-ROS fusion polypeptide or deletion ROS polypeptide, and to monitor regulation of SLC34A2-ROS fusion polypeptide, CD74-ROS fusion polypeptide or deletion ROS polypeptide levels during therapeutic intervention. In one preferred embodiment, hybridization with PCR probes which are capable of detecting polynucleotide sequences, including genomic sequences, encoding SLC34A2-ROS fusion polypeptide, CD74-ROS fusion polypeptide or FIG-ROS fusion polypeptide or closely related molecules, may be used to identify nucleic acid sequences that encode genetically altered ROS polypeptide. The construction and use of such probes is known to those skilled in the art and described in United States Patent Publication US2010/0221737.

The specificity of the probe, whether it is made from a highly specific region, e.g., unique nucleotides in the fusion junction, or a less specific region, e.g., the 3′ coding region, and the stringency of the hybridization or amplification (maximal, high, intermediate, or low) will determine whether the probe identifies only naturally occurring sequences encoding genetically altered ROS polypeptides, alleles, or related sequences. Probes may also be used for the detection of related sequences, and should preferably contain at least 50% of the nucleotides from any of the genetically altered ROS polypeptide encoding sequences.

A SLC34A2-ROS fusion polynucleotide, CD74-ROS fusion polynucleotide or deletion ROS polynucleotide may be used in Southern or northern analysis, dot blot, or other membrane-based technologies; in PCR technologies; or in dip stick, pin, ELISA or chip assays utilizing fluids or tissues from patient biopsies to detect genetically altered ROS polypeptide expression. Such qualitative or quantitative methods are well known in the art. In a particular aspect, the nucleotide sequences encoding genetically altered ROS polypeptides may be useful in assays that detect activation or induction of various cancers, including cancers of the lung including NSCLC. Genetically altered ROS polynucleotides may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable control sample, the nucleotide sequences have hybridized with nucleotide sequences in the sample, and the presence of altered levels of nucleotide sequences encoding SLC34A2-ROS fusion polypeptide, CD74-ROS fusion polypeptide or deletion ROS polypeptide in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease characterized by expression of genetically altered ROS polypeptide, a normal or standard profile for expression is established. This may be accomplished by combining body fluids or cell extracts taken from normal subjects, either animal or human, with a sequence, or a fragment thereof, which encodes SLC34A2-ROS fusion polypeptide, CD74-ROS fusion polypeptide or deletion ROS polypeptide (e.g., FIG-ROS fusion polypeptide), under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease.

Once disease is established and a treatment protocol is initiated, hybridization assays may be repeated on a regular basis to evaluate whether the level of expression in the patient begins to approximate that which is observed in the normal patient. The results obtained from successive assays may be used to show the efficacy of treatment over a period ranging from several days to months.

Additional diagnostic uses for genetically altered ROS polynucleotides may involve the use of polymerase chain reaction (PCR), another preferred assay format that is standard to those of skill in the art. (See, e.g., MOLECULAR CLONING, A LABORATORY MANUAL, 2nd. edition, Sambrook, J., Fritsch, E. F. and Maniatis, T., eds., Cold Spring Harbor Laboratory Press, Cold Spring Harbor, N.Y. (1989)). PCR oligomers may be chemically synthesized, generated enzymatically, or produced from a recombinant source. Oligomers will preferably consist of two nucleotide sequences, one with sense orientation (5′ to 3′) and another with antisense (3′ to 5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Methods which may also be used to quantitate the expression of SLC34A2-ROS fusion polypeptide, CD74-ROS fusion polypeptide or deletion ROS polypeptide include radiolabeling or biotinylating nucleotides, coamplification of a control nucleic acid, and standard curves onto which the experimental results are interpolated (Melby et al., J. Immunol. Methods, 159: 235-244 (1993); Duplaa et al. Anal. Biochem. 229-236 (1993). The speed of quantitation of multiple samples may be accelerated by running the assay in an ELISA format where the oligomer of interest is presented in various dilutions and a spectrophotometric or colorimetric response gives rapid quantitation.

Genetically altered ROS polynucleotides may be used to generate hybridization probes which are useful for mapping the naturally occurring genomic sequence. The sequences may be mapped to a particular chromosome or to a specific region of the chromosome using well known techniques. Such techniques include fluorescence in-situ hybridization (FISH), FACS, or artificial chromosome constructions, such as yeast artificial chromosomes, bacterial artificial chromosomes, bacterial P1 constructions or single chromosome cDNA libraries, as reviewed in Price, C. M., Blood Rev. 7: 127-134 (1993), and Trask, B. J., Trends Genet. 7: 149-154 (1991). In one non-limiting embodiment, FISH is employed (as described in Verma et al. HUMAN CHROMOSOMES: A MANUAL OF BASIC TECHNIQUES, Pergamon Press, New York, N.Y. (1988) and may be correlated with other physical chromosome mapping techniques and genetic map data. Examples of genetic map data can be found in the 1994 Genome Issue of Science (265: 1981f). Correlation between the location of the gene encoding SLC34A2-ROS fusion polynucleotide, CD74-ROS fusion polynucleotide or deletion ROS polynucleotide on a physical chromosomal map and a specific disease, or predisposition to a specific disease, may help delimit the region of DNA associated with that genetic disease. The nucleotide sequences may be used to detect differences in gene sequences between normal, carrier, or affected individuals.

In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques.

It shall be understood that all of the methods (e.g., PCR and FISH) that detect genetically altered ROS polynucleotides may be combined with other methods that detect genetically altered ROS polynucleotides or genetically altered ROS polypeptides. For example, detection of a SLC34A2-ROS polynucleotide in the genetic material of a biological sample (e.g., in a circulating tumor cell) may be followed by Western blotting analysis or immunohistochemistry (IHC) analysis of the proteins of the sample to determine if the SLC34A2-ROS polynucleotide was actually expressed as a SLC34A2-ROS polypeptide in the biological sample. Such Western blotting or IHC analyses may be performed using an antibody that specifically binds to the polypeptide encoded by the detected SLC34A2-ROS polynucleotide, or the analyses may be performed using antibodies that specifically bind either to full length SLC34A2 (e.g., bind to the N-terminus of the protein) or to full length ROS (e.g., bind an epitope in the kinase domain of ROS). Such assays are known in the art (see, e.g., U.S. Pat. No. 7,468,252).

ROS Kinase Therapeutics

It has been shown that genetically altered ROS polypeptides occur in at least one subgroup of human NSCLC (See, Rikova, et al., Cell 131:1190-1203 (2007)). Accordingly, the progression of a mammalian cancer (e.g. NSCLC) in which at least one ROS fusion protein (e.g. SLC34A2-ROS fusion protein) is expressed may be inhibited, in vivo, by inhibiting the activity of ROS kinase in such cancer or by inhibiting the expression of ROS kinase in such cancer. ROS activity in cancers characterized by expression of a mutant ROS kinase may be inhibited by contacting the cancer (e.g. a tumor) with a ROS kinase therapeutic.

A ROS kinase therapeutic may be any composition comprising at least one compound, biological or chemical, which inhibits, directly or indirectly, the expression and/or activity of ROS kinase in vivo, including the ROS kinase inhibitor compounds described below. Such compounds include therapeutics that act directly on ROS kinase itself, or on proteins or molecules that modify the activity of ROS, or that act indirectly by inhibiting the expression of ROS. Such compositions also include compositions comprising only a single ROS kinase inhibiting compound, as well as compositions comprising multiple therapeutics (including those against other RTKs), which may also include a non-specific therapeutic agent like a chemotherapeutic agent or general transcription inhibitor.

Small Molecule ROS Kinase Inhibitors

ROS kinase therapeutics useful in the practice of the methods of the invention are small molecule ROS kinase inhibitors. Small molecule kinase inhibitors are a class of molecules that typically inhibit the activity of their target enzyme by specifically, and often irreversibly, binding to the catalytic site of the enzyme, and/or binding to an ATP-binding cleft or other binding site within the enzyme that prevents the enzyme from adopting a conformation necessary for its activity. Small molecule ROS kinase inhibitors may be rationally designed using X-ray crystallographic or computer modeling of ROS kinase three-dimensional structure, or may found by high throughput screening of compound libraries for inhibition of ROS. Such methods are well known in the art, and have been described. Specificity of ROS inhibition may be confirmed, for example, by examining the ability of such compounds to inhibit ROS activity, but not other kinase activity, in a panel of kinases, and/or by examining the inhibition of ROS activity in a biological sample comprising tumor cells that are known to or modified to express a ROS fusion protein.

Examples of small molecule ROS inhibitors shown, herein, to be useful as ROS kinase therapeutics include amino-pyridine and amino-pyrazine compounds of the type disclosed in U.S. Pat. No. 7,230,098, U.S. Pat. No. 7,858,643, and WO 2006/021881 each of which is incorporated herein by reference in their entirety for all they disclose. Specifically, amino-pyridine and amino-pyrazine compounds useful in connection with the present invention as ROS kinase therapeutics include compounds of the general formula:

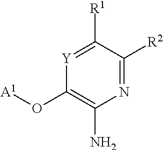

or pharmaceutically acceptable salts thereof, where Y, R¹, R² and A¹ have the general meanings as described in U.S. Pat. No. 7,230,098. More specifically, amino-pyridine and amino-pyrazine compounds useful in connection with the present invention as ROS kinase therapeutics include compounds of the general formula:

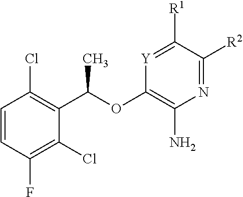

or pharmaceutically acceptable salts thereof, where Y, R¹, and R² have the general meanings as described in U.S. Pat. No. 7,858,643. Amino-pyridine and amino-pyrazine compounds of the type described above have been shown to be ROS kinase inhibitors and are thus useful as ROS kinase therapeutics in connection with the present invention. Specifically, where a cancer is shown to be positive for a genetically altered ROS kinase (e.g., SLC34A2-ROS, CD74-ROS or FIG-ROS), such compounds can be administered to a patient in need of treatment of cancer.

One particularly preferred compound is the compound 3-[(R)-1-(2,6-dichloro-3-fluoro-phenyl)-ethoxy]-5-(1-piperidin-4-yl-1H-pyrazol-4-yl)-pyridin-2-ylamine (crizotinib), represented by the formula 1:

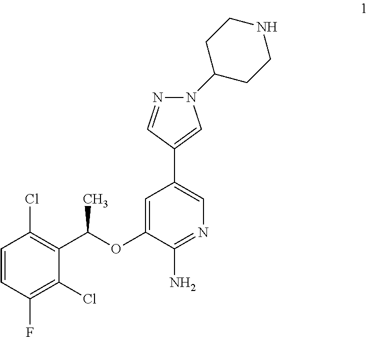 [SMILES: C[C@@H](Oc1cc(-c2cnn(C3CCNCC3)c2)cnc1N)c1c(Cl)ccc(F)c1Cl]

the preparation of which is described in U.S. Pat. No. 7,858,643. (See also, McDermott, U. et al., Proc. Natl. Acad. Sci. 104, 19936-19941 (2007)). The compound of formula 1 is disclosed in International Patent Publication WO 2006/021884 and United States Patent Application No. 2006/0046991, the contents of each of which are herein incorporated by reference in their entirety. Additionally, the racemate of the compound of formula 1 is disclosed in International Patent Publication WO 2006/021881 and United States Patent Application No. 2006/0128724, the contents of each of which are herein incorporated by reference in their entirety.

Originally designed as a c-Met/HGFR inhibitor, crizotinib has been shown herein to be active against ROS kinase, and thus active against the ROS fusion proteins and ROS deletion proteins described herein. Crizotinib was evaluated for its effect on ROS catalytic activity in both enzyme and cell-based assays. The data provided herein demonstrate crizotinib to be a potent ATP-competitive inhibitor of recombinant, human ROS-1 kinase (catalytic domain).

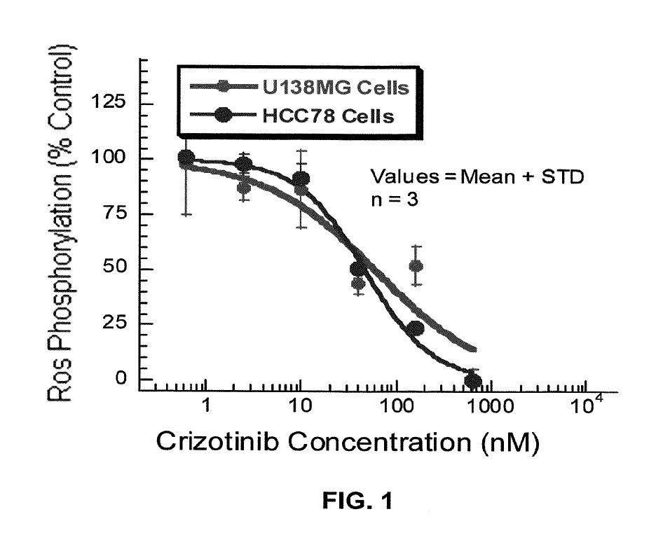

A ROS-1 enzyme assay described below provided a mean Ki value of 0.097 nM (n=4). Crizotinib dose-dependently inhibited ROS phosphorylation in HCC78 cells that exhibit a 4p15, 6q22 chromosomal translocation event resulting in the expression of a constitutively active SLC34A2-ROS fusion protein (Rikova et al. (2007)) with a mean IC₅₀ value of 41 nM (n=11) (Table 1, FIG. 1). Crizotinib also dose-dependently inhibited ROS phosphorylation in U138MG human glioblastoma cells harboring FIG-ROS fusion (Charest et al. (2003)) with a mean IC₅₀ value of 49 nM (n=2) (Table 1, FIG. 1).

In a panel of 3T3 cell lines that were engineered to express various ROS-fusion proteins, including CD74-ROS, FIG-ROS(S), FIG-ROS(L), SLC34A2-ROS(S), and SLC34A2-ROS(L), crizotinib inhibited ROS phosphorylation with IC₅₀ values ranging from 3.4 nM to 36 nM (Table 1).

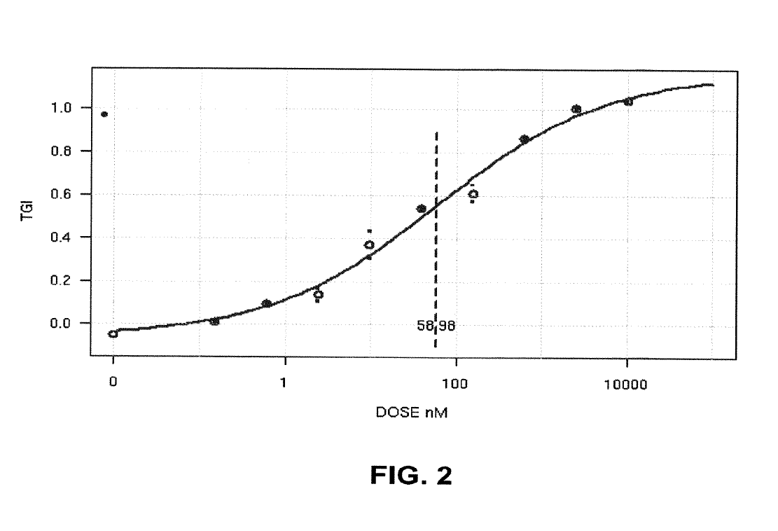

Crizotinib was also evaluated for its effect on cell viability of HCC78 that exhibit a 4p15, 6q22 chromosomal translocation event resulting in the expression of a constitutively active SLC34A2-ROS fusion protein (Rikova et al. (2007)). Crizotinib demonstrated concentration dependent inhibition of HCC78 cell viability (FIG. 2). The IC₅₀ value calculated for inhibition of HCC78 cell viability was approximately 59 nM. These results suggest that HCC78 cells are dependent on the ROS fusion for cell growth and viability and that crizotinib is a potent inhibitor of ROS-dependent cell growth and viability.

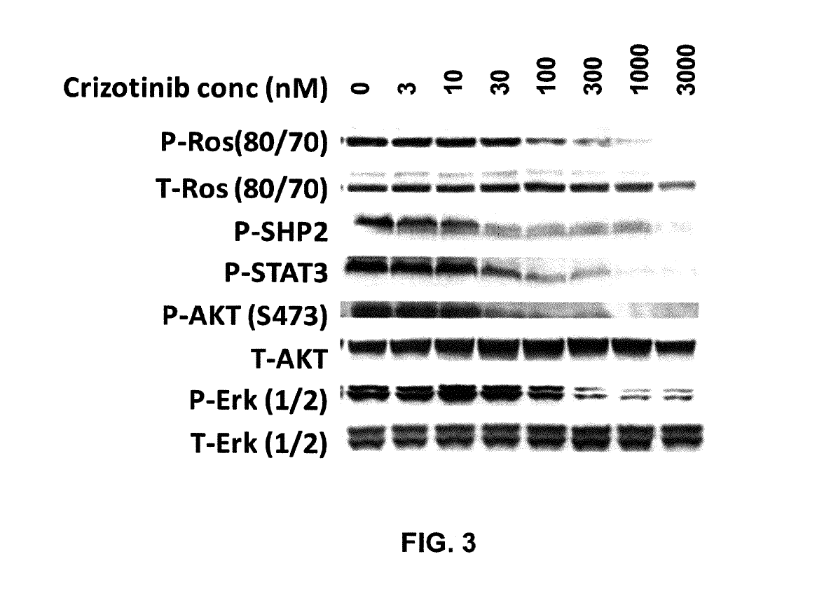

At the molecular level, the constitutively activated ROS fusion kinase induces phosphorylation of multiple tyrosine residues at the intracellular region that regulates RTK catalytic activity and docking of regulatory substrates. Crizotinib was evaluated for its ability to inhibit SLC34A2-ROS dependent signaling pathways in HCC78 human NSCLC cells in order to gain further understanding of the anti-tumor mechanism-of-action and to confirm that inhibition of ROS kinase activity correlates with downstream signal transduction. Crizotinib dose dependently inhibited ROS phosphorylation (activation loop), as well as the downstream adaptor or signaling molecules including SHP2, STAT3, AKT and ERK1/2 in the HCC78 cells in vitro (FIG. 3). These results demonstrate a correlation between key signaling pathways and efficacious doses of crizotinib.

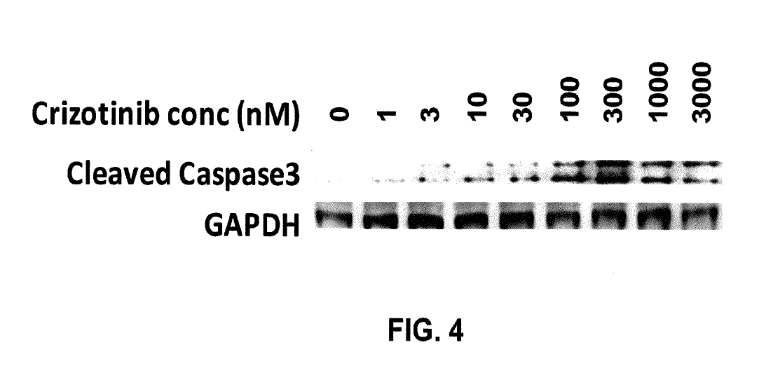

Crizotinib was further evaluated for its ability to induce cell apoptosis in HCC78 human NSCLC cells. Crizotinib demonstrated dose-dependent induction of activated caspase-3 levels in the HCC78 NSCLC cells (FIG. 4), demonstrating that increased apoptosis also correlated with efficacious dose levels.

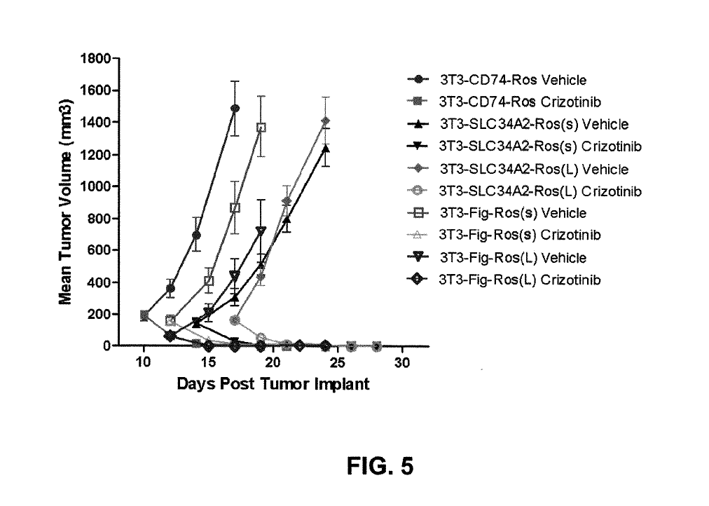

The antitumor efficacy of crizotinib was evaluated in a panel of ROS fusion engineered tumor xenograft models. Tumor xenografts representative of human cancer indications in which ROS chromosomal translocations have been implicated were engineered in NIH3T3 cells, including CD74-ROS, long and short variants of SLC34A2-ROS identified in human NSCLC, and long and short variants of Fig-ROS identified in human NSCLC, glioblastoma and cholangiocarcinoma (Rimkunas et al. (2012); Gu et al. (2011)). Crizotinib demonstrated significant cytoreductive effects in all of the 3T3-ROS engineered tumor models with a dosing regimen of 75/mg PO BID (FIG. 5).

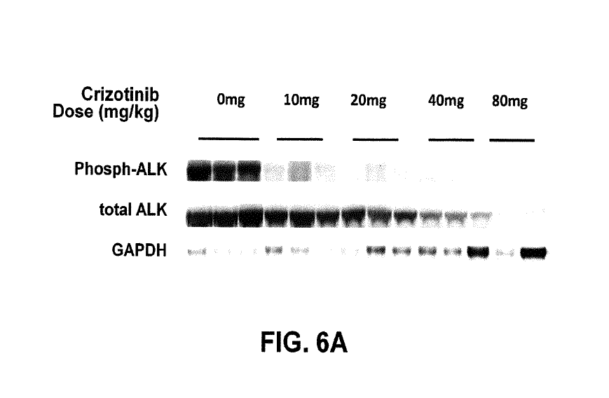

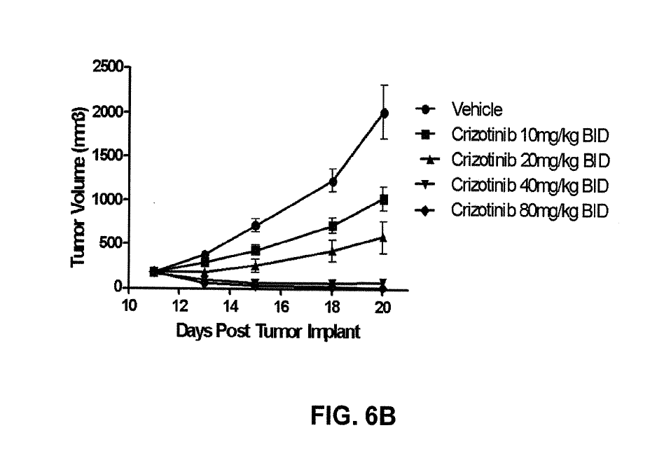

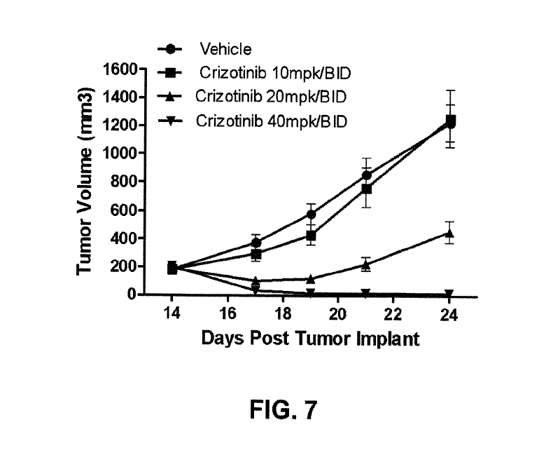

The ability of crizotinib to inhibit ROS phosphorylation and tumor growth in vivo was evaluated in 3T3-CD74-ROS and the 3T3-SLC34A2-ROS(L) xenograft models in nude mice. Crizotinib demonstrated dose-dependent inhibition in tumor growth in 3T3-CD74-Ros tumor xenograft at doses of 160 mg/kg/day (80 mg/kg BID), 80 mg/kg/day (40 mg/kg BID), 40 mg/kg/day (20 mg/kg BID) and 20 mg/kg/day (10 mg/kg BID) (FIG. 6B). crizotinib also demonstrated significant inhibition of ROS phosphorylation in the 3T3-CD74-Ros tumors across all treatment groups (FIG. 6A). Similar antitumor efficacy by crizotinib was observed in the 3T3-SLC34A2-ROS(L) xenograft model (FIG. 7).

Routes of Administration and Dosage Forms

Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a pharmaceutically acceptable carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste-masking agents.

Exemplary tablets contain up to about 80 wt % drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Solid formulations for oral administration may be formulated to be immediate and/or modified release. Modified release formulations include delayed-, sustained-, pulsed-, controlled-, targeted and programmed release.

Suitable modified release formulations are described in U.S. Pat. No. 6,106,864. Details of other suitable release technologies such as high energy dispersions and osmotic and coated particles can be found in Verma et al, Pharmaceutical Technology On-line, 25(2), 1-14 (2001). The use of chewing gum to achieve controlled release is described in WO 00/35298. The disclosures of these references are incorporated herein by reference in their entireties.

Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including micro needle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

Topical Administration

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J Pharm Sci, 88 (10), 955-958 by Finnin and Morgan (October 1999). Other means of topical administration include delivery by electroporation, iontophoresis, phonophoresis, sonophoresis and micro needle or needle-free (e.g. Powderject™, Bioject™, etc.) injection. The disclosures of these references are incorporated herein by reference in their entireties.

Formulations for topical administration may be formulated to be immediate and/or modified release. Modified release formulations include delayed-, sustained-, pulsed-, controlled-, targeted and programmed release.

Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may include a bioadhesive agent, for example, chitosan or cyclodextrin.

The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 μg to 20 mg of the compound of the invention per actuation and the actuation volume may vary from 1 μL to 100 μL. A typical formulation includes a compound of the invention, propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing a desired mount of the compound of the invention. The overall daily dose may be administered in a single dose or, more usually, as divided doses throughout the day.

Rectal/Intravaginal Administration

Compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

Ocular Administration

Compounds of the invention may also be administered directly to the eye or ear, typically in the form of drops of a micronized suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

Other Technologies

Compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins, examples of which may be found in PCT Publication Nos. WO 91/11172, WO 94/02518 and WO 98/55148, the disclosures of which are incorporated herein by reference in their entireties.

Dosage

The amount of the active compound administered will be dependent on the subject being treated, the severity of the disorder or condition, the rate of administration, the disposition of the compound and the discretion of the prescribing physician. However, an effective dosage is typically in the range of about 0.001 to about 100 mg per kg body weight per day, preferably about 0.01 to about 35 mg/kg/day, in single or divided doses. For a 70 kg human, this would amount to about 0.07 to about 7000 mg/day, preferably about 0.7 to about 2500 mg/day. In some instances, dosage levels below the lower limit of the aforesaid range may be more than adequate, while in other cases still larger doses may be used without causing any harmful side effect, with such larger doses typically divided into several smaller doses for administration throughout the day.

Kit-of-Parts

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions. Thus the kit of the invention includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions or instructions for administration and may be provided with a memory aid. Such directions or instructions may be in the form of a “label” or pamphlet. Further such directions or instructions may contain information relating to diagnostic testing to determine whether a cancer is ROS positive or whether a patient is ROS positive.

### EXAMPLES

### In-Vitro Assays

Materials and Methods

In-Vitro Methods

ROS-1 Enzymatic Assay

Inhibition of ROS-1 enzyme was measured using a microfluidic mobility shift assay. The reactions were conducted in 50 μL volumes in 96-well plates, and contained 0.25 nM recombinant human ROS-1 catalytic domain (aa 1883-2347), GST-tagged (Invitrogen Inc), 1.5 μM phosphor-acceptor peptide, 5′FAM-KKSRGDYMTMQIG-CONH₂ (Caliper LifeSciences), test compound (11-dose 3-fold serial dilutions, 2% DMSO final) or DMSO only, 1 mM DTT, 0.002% Tween-20 and 5 mM MgCl₂ in 25 mM Hepes, pH 7.1, and were initiated by addition of ATP (56 μM final concentration, ˜Km level) following a 20-min pre-incubation. The reactions were incubated for 1 hour at room temperature, then stopped by the addition of 0.1 M EDTA (pH 8). The extent of reaction completion (˜5% conversion with DMSO) was determined after electrophoretic separation of the fluorescently labeled peptide substrate and phosphorylated product on a LabChip EZ Reader II (Caliper LifeSciences). Ki values for each trial was calculated by fitting the % conversion to the equation for competitive inhibition using non-linear regression method (GraphPad Prism, GraphPad Software, San Diego, Calif.) and experimentally measured ATP Kₘ=56 μM. A panel of four trials gave an average Ki value of 0.097 nM.

Cell Lines

HCC78 cells are a human non-small cell lung carcinoma cell line established from the pleural effusion of a 65-year-old man with adenocarcinoma of the lung, typed as non-small cell lung carcinoma. HCC78 cells were purchased from DSMZ cell bank (Braunschweig, Germany). U138 cells and NIH3T3 cells were purchased from American Tissue Culture Corporation TCC.

NIH3T3-ROS Fusion Cell Line Generation

The NIH3T3 ROS fusion engineered cell lines were generated in house. ROS fusion variants SLC34A2-ROS (L), SLC34A2-ROS (S), CD74-ROS (L), FIG-ROS (L) and FIG-ROS (S) were cloned into the retroviral vector pMSCV puro (Clontech). The retroviruses carrying EML4-ALK genes were produced in 293T cells by co-transfection with the pMSCV vectors and the packaging plasmid pC10A1. The retroviral supernatants were used to transduce NIH3T3 cells and pooled populations were selected with 2 μg/ml puromycin for 5 days and verified by DNA sequencing prior to use in subsequent experiments.

Cellular Kinase Phosphorylation Assays

Cellular assays (i.e., ELISA or immunoblot) used to directly determine the ability of crizotinib to inhibit ligand-dependent or constitutive kinase phosphorylation were performed using a variety of serum-starved cells.

Cell-Based Phospho-ROS ELISA Assay

A panel of cell lines harboring various kinds of ROS fusion was used to determine the potency of crizotinib on ROS phosphorylation. The cells were plated at a density of 20,000 cells/well in 100 μl of the growth media in 96-well plates. The ROS fusion negative cells wells were used as background. Plated cells were allowed to adhere overnight. The following day, growth media was removed and cells were cultured in serum-free media (with 0.04% BSA). Serial dilutions of crizotinib were performed, appropriate controls or designated concentrations of crizotinib were added to each well, and cells were incubated at 37° C. for 1 hour. Cell lysates were generated and the total phospho-tyrosine levels of SLC34A2-ROS in HCC78 cells were determined by using the PathScan® Phospho-Ros (panTyr) Sandwich ELISA Kit (Cell Signaling, Cat#: 7093) as described in the manufacturer's protocol. The EC₅₀ values were calculated by concentration-response curve fitting utilizing a four-parameter analytical method.

Cell-Based Phospho-ROS ELISA Assay for SLC34A2-ROS

HCC78 cells harboring SLC34A2-ROS fusion were used to determine the potency of crizotinib on ROS phosphorylation. HCC78 cells were plated at a density of 20,000 cells/well in 100 μl of RPMI media with 10% FBS and penicillin/streptomycin in 96-well plates. The no cell wells were used as background. Plated cells were allowed to adhere overnight. The following day, growth media was removed and cells were cultured in serum-free media (with 0.04% BSA). Serial dilutions of crizotinib were performed, appropriate controls or designated concentrations of crizotinib were added to each well, and cells were incubated at 37° C. for 1 hour. Cell lysates were generated and the total phospho-tyrosine levels of SLC34A2-ROS in HCC78 cells were determined by using the PathScan® Phospho-Ros (panTyr) Sandwich ELISA Kit (Cell Signaling, Cat#: 7093) as described in the manufacturer's protocol. The IC₅₀ values were calculated by concentration-response curve fitting utilizing a four-parameter analytical method. The cell-based phospho-ROS ELISA assay provided a mean IC₅₀ value of 45 nM (n=8).

Immunoblotting

Immunoblotting methods were also used to determine relative kinase phosphorylation status and total protein levels in the HCC78 cells and 3T3-CD74-ROS tumor lysates for the protein of interest. For in vitro study, the HCC78 cells were treated with various dose levels of crizotinib for three hours. The cells were lysed in the cold 1X Cell Lysis Buffer (Cell Signaling Technologies, Boston Mont.).

For in vivo study, tumor bearing mice were treated with crizotinib 75 mg/kg PO BID for 10 days. At the end of study, tumors were resected after 7 hours following the last dose. The resected tumors were snap frozen on dry ice, pulverized using a liquid nitrogen-cooled cryomortar and pestle, and lysed in cold 1X Cell Lysis Buffer (Cell Signaling Technologies, Boston Mont.). The Proteins were extracted from cell and tumor lysates and protein concentrations were determined using a BSA assay (Pierce, Rockford, Ill.). Extracted protein samples from both cell and tumor lysates were separated by SDS-PAGE, transferred to nylon membranes, and immunoblotting hybridizations for the proteins of the interest were performed with the following antibodies.

Antibodies utilized in immunoblotting studies were all from Cell Signaling Technology (Danvers, Mass., United States) and listed as follows: anti-total ROS (catalog#: 3266), anti-phospho ROS (catalog#: 3078), anti-phospho SHP2 (catalog#: 5431), anti-phospho STAT3 (catalog#: 9131), anti-total AKT (catalog#: 9272), anti-phospho-AKT S473 (catalog#: 4161), anti-total-MAPK44/42 (catalog#: 9102), anti phospho-MAPK44/42 (catalog#: 4370), cleaved Caspapse-3 (catalog#: 9661).

Cell Viability, Proliferation and Survival Assays

Cell Viability Assay

Cultured HCC78 cells were adapted to RPMI growth medium (Invitrogen, Carlsbad, Calif.) with 10% FBS and penicillin/streptomycin (Invitrogen) whenever possible to standardize screening. Some cells requiring special media were grown in vendor recommended media. Cells were trypsinized and seeded at a density of 3000-5000 cells/well into 96-well plates (Corning Costar #3904 plates, Kennebunk, Me.) and allowed to adhere overnight. The following day, cells were treated with single agent drug administered in nine serial concentrations in duplicates (progressively decreasing from 10 μM to 152 pM by a 4-fold ratio yielding a full sigmoidal curve). After an additional 3-5 days incubation at 37° C. (until cell confluency reached ˜70-80%), 1/5 of manufacturer's recommended volume of Cell Titer Glo (Promega, Madison, Wis.) was added to indirectly measure cell viability/proliferation using an Envision multi-reader (Perkin-Elmer, Waltham, Mass.). Baseline cell count readings were also taken from cell plates one-day after cell seeding and prior to drug treatment. Baseline count was subtracted from final cell count and plotted with PRISM (Graphpad, La Jolla, Calif.) or XLFIT (IDBS, Surrey, UK). The IC₅₀ value calculated for inhibition of HCC78 cell viability was approximately 59 nM.

Cell Proliferation/Survival Assay

The cells were seeded in 96 well plates at low density in growth media (media supplemented with 2%, 5% or 10% fetal bovine serum-FBS) and cultured overnight at 37° C. The following day, serial dilutions of crizotinib or appropriate controls were added to the designated wells, and cells were incubated at 37° C. for 72 hours. A Cell Titer Glo assay (Promega, Madison, Wis.) was then performed to determine the relative cell numbers. EC₅₀ values were calculated by concentration-response curve fitting utilizing a four-parameter analytical method.

In Vivo Methods

Subcutaneous Xenograft Models in Athymic Mice

Female nu/nu mice (5-8 weeks old) were obtained from Charles River (Wilmington, Mass.). Animals were maintained under clean room conditions in sterile filter top cages with Alpha-Dri/bed-o-cob comb bedding housed on HEPA-filtered ventilated racks. Animals received sterile rodent chow and water ad libitum. The designated cells for implantation into athymic mice were harvested and pelleted by centrifugation at 450×g for 5-10 minutes. The cell pellets were washed once and re-suspended in serum-free medium. The cells were supplemented with 50% Matrigel (BD Biosciences, San Jose Calif.) to facilitate tumor take. Cells (5×106 in 100 μL) were implanted SC into the hind flank region of the mouse and allowed to grow to the designated size prior to the administration of compound for each experiment. Tumor size was determined by measurement with electronic calipers and tumor volume was calculated as the product of its length×width²×0.4.

Data and Results

### Example 1

### Inhibition of ROS1 Kinase Activity in Biochemical Enzyme Assays by Crizotinib

Crizotinib was evaluated for its effect on ROS catalytic activity in both enzyme and cell-based assays. Crizotinib was demonstrated to be a potent ATP-competitive inhibitor of recombinant, human ROS1 kinase (catalytic domain) with a mean Ki value of 0.097 nM (n=4).

### Example 2

### Kinase Activity of Crizotinib in Cell-Based Assays

Crizotinib dose-dependently inhibited ROS phosphorylation with a mean IC₅₀ value of 41 nM (n=11) in the HCC78 cells that exhibit a 4p15, 6q22 chromosomal translocation event resulting in the expression of a constitutively active SLC34A2-ROS fusion protein (Rikova et al. (2007)) in these cells (Table 1, FIG. 1).

Crizotinib also inhibited ROS phosphorylation with a mean IC₅₀ value of 49 nM (n=2) in the U138MG human glioblastoma cells harboring FIG-ROS fusion (Charest, et al. (2003)) (Table 1, FIG. 1).

In a panel of 3T3 cell lines that were engineered to express various ROS-fusion proteins, crizotinib inhibited ROS phosphorylation with IC₅₀ values ranging from 3.4 nM to 36 nM in these cells (Table 1).

TABLE 1

| | IC₅₀ (nM) | |
|---|---|---|
| | Mean + | |
| Cell-based ROS1 Kinase Phosphorylation Assays | STD | n |
| Endogenous SLC34A2-ROS phosphorylation in HCC78 | 41 ± 14 | 11 |
| human NSCLC cells | | |
| Endogenous FIG-ROS phosphorylation in U138MG | 49 ± 18 | 2 |
| human glioma cells | | |
| Engineered CD74-ROS phosphorylation in 3T3-CD74- | 3.4 ± 2.4 | 4 |
| ROS cells | | |
| Engineered FIG-ROS(S) phosphorylation in 3T3-FIG- | 19 ± 11 | 4 |
| ROS(S) cells | | |
| Engineered FIG-ROS(L) phosphorylation in 3T3-FIG- | 8.7 ± 0.6 | 3 |
| ROS(L) cells | | |
| Engineered SLC34A2-ROS(S) phosphorylation in 3T3- | 19 + 15 | 2 |
| SLC34A2-ROS(S) cells | | |
| Engineered SLC34A2-ROS(L) phosphorylation in 3T3- | 36 | 1 |
| SLC34A2-ROS(L) cells | | |

### Example 3

### Inhibition of ROS Mediated Signal Transduction and Induction of Cell Apoptosis in the HCC78 Human NSCLC Cells In Vitro

Crizotinib was evaluated for its ability to inhibit SLC34A2-ROS dependent signaling pathways in the HCC78 cells.

As illustrated in the immunoblot in FIG. 3, crizotinib dose dependently inhibited ROS phosphorylation (activation loop), as well as the downstream adaptor or signaling molecules including SHP2, STAT3, AKT and ERK1/2 following 3 hours of drug treatment in the HCC78 cells in vitro. These data demonstrated a correlation between key signaling pathways and efficacious doses of crizotinib.

Crizotinib was evaluated for its dose-dependent modulation of the caspase-3 marker of apoptosis utilizing Western Blot analysis. Following 3-hour of drug treatment, a significant dose-dependent induction of activated caspase-3 levels was observed in the HCC78 NSCL cells (FIG. 4) indicating that increased apoptosis also correlated with efficacious dose levels.

### Example 4

### Cytoreductive Effect of Crizotinib Following Oral Administration in a Panel of Oncogenic ROS Fusion Variants Engineered Xenograft Tumor Models in Nude Mice

The antitumor efficacy of crizotinib was evaluated in a panel of ROS fusion engineered tumor xenograft models in the NIH3T3 cells representative of human cancer indications in which ROS chromosomal translocation is implicated, including CD74-ROS, two forms of SLC34A2-ROS that were identified in human NSCLC, and two forms of FIG-ROS that were identified in human NSCLC, glioblastoma and cholangiocarcinoma (Rimkunas et al. (2012) Clin Cancer Res. June 1. [Epub ahead of print]); Gu et al. (2011) PLoS One. 6(1):e15640).

Crizotinib demonstrated significant cytoreductive effects in all of the five 3T3-ROS engineered tumor models that harbor human oncogenic ROS fusion variants with a dosing regimen of 75/mg PO BID as shown in FIG. 5. The mice started receiving crizotinib treatment when the tumor volume reached ˜200 mm³, and the tumors regressed rapidly to the size of 5 to 10 mm³ in about 4 to 5 days of drug treatment. The control tumors reached the size of 1500 mm³ in ˜7 days after dosing start, and the average crizotinib treatment time for this study was ˜10 days.

### Example 5

### Dose-Dependent Inhibition of ROS Phosphorylation and Tumor Growth in the 3T3-CD74-ROS and the 3T3-SLC34A2-ROS(L) Xenograft Models in Nude Mice by Crizotinib

To evaluate pharmacodynamic inhibition of ROS kinase activity and tumor growth by crizotinib, 3T3-CD74-ROS tumor xenograft study in nude mice with oral BID dosing at multiple dose levels was conducted. The tumor volume was measured utilizing electronic Vernier calipers throughout the study and tumors samples were harvested at 7-hour following oral administration of crizotinib for 10 days (steady-state). ROS phosphorylation status in tumors was quantitated by ELISA.

Crizotinib demonstrated dose-dependent inhibition in tumor growth as shown in FIG. 6B. Tumor regressions of 94% and 61% were observed in the 160 mg/kg/day group (80 mg/kg BID) and the 80 mg/kg/day group (40 mg/kg BID) respectively, and 78% and 54% tumor growth inhibition were observed in the 40 mg/kg/day group (20 mg/kg BID) and the 20 mg/kg/day group (10 mg/kg BID), respectively.

At 7 hours post last crizotinib oral administration, significant inhibition of ROS phosphorylation in the 3T3-CD74-ROS tumors was observed across all the treatment groups (FIG. 6A).

A similar degree of antitumor efficacy by crizotinib in the 3T3-SLC34A2-ROS(L) model was also observed (FIG. 7).

### Example 6

### Synthesis of the Compound of Formula 1 (Crizotinib)

PLE is an enzyme produced by Roche and sold through Biocatalytics Inc. as a crude esterase preparation from pig liver, commonly known as PLE-AS (purchased from Biocatalytics as ICR-123, sold as an ammonium sulfate suspension). The enzyme is classified in the CAS registry as a “carboxylic-ester hydrolase, CAS no. 9016-18-6”. The corresponding enzyme classification number is EC 3.1.1.1. The enzyme is known to have broad substrate specificity towards the hydrolysis of a wide range of esters. The lipase activity is determined using a method based on hydrolysis of ethyl butyrate in a pH titrator. 1 LU (lipase unit) is the amount of enzyme which liberates 1 μmol titratable butyric acid per minute at 22° C., pH 8.2. The preparation reported herein (PLE-AS, as a suspension) is usually shipped as an opaque brown-green liquid with a declared activity of >45 LU/mg (protein content around 40 mg/mL).

### (1S)-1-(2,6-dichloro-3-fluorophenyl)ethanol

(1S)-1-(2,6-dichloro-3-fluorophenyl)ethanol, shown as compound (S-1) in the schemes below, was prepared by a combination of enzymatic hydrolysis of racemic 1-(2,6-dichloro-3-fluorophenyl)ethyl acetate, esterification and chemical hydrolysis with inversion according to Scheme B. Racemic 1-(2,6-dichloro-3-fluorophenyl)ethyl acetate (compound A2) was prepared according to Scheme A.

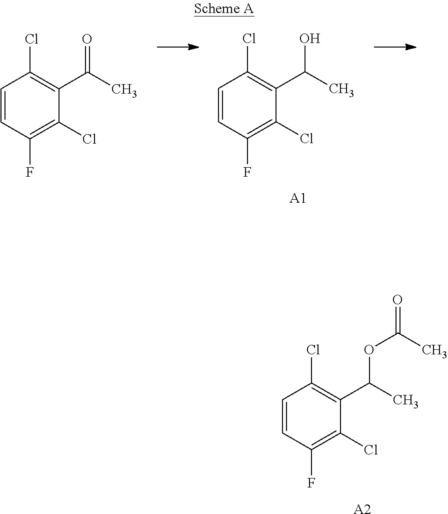

### 1-(2,6-dichloro-3-fluorophenyl)ethanol (A1)

Sodium borohydride (90 mg, 2.4 mmol) was added to a solution of 2′,6′-dichloro-3′-fluoro-acetophenone (Aldrich, catalog #52,294-5) (207 mg, 1 mmol) in 2 mL of anhydrous CH₃OH. The reaction mixture was stirred at room temperature for 1 h then was evaporated to give a colorless oil residue. The residue was purified by flash chromatography (eluting with 0→10% EtOAc in hexanes) to give compound A1 as a colorless oil (180 mg; 0.88 mmol; 86.5% yield); MS (APCI) (M-H)⁻ 208; 1 H NMR (400 MHz, chloroform-D) δ ppm 1.64 (d, J=6.82 Hz, 3H) 3.02 (d, J=9.85 Hz, 1H) 6.97-7.07 (m, 1H) 7.19-7.33 (m, 1H).

### 1-(2,6-dichloro-3-fluorophenyl)ethyl acetate (A2)

Acetic anhydride (1.42 mL, 15 mmol) and pyridine (1.7 mL, 21 mmol) were added sequentially to a solution of compound A1 (2.2 g, 10.5 mmol) in 20 mL of CH₂Cl₂. The reaction mixture was stirred at room temperature for 12 h and then evaporated to give a yellowish oil residue. The residue was purified by flash chromatography (eluting with 7→9% EtOAc in hexanes) to give compound A2 as a colorless oil (2.26 g; 9.0 mmol; 85.6% yield); 1H NMR (400 MHz, chloroform-D) δ ppm 1.88 (d, J=6.82 Hz, 3H) 2.31 (s, 3H) 6.62 (q, J=6.82 Hz, 1H) 7.25 (t, J=8.46 Hz, 1H) 7.49 (dd, J=8.84, 5.05 Hz, 1H).

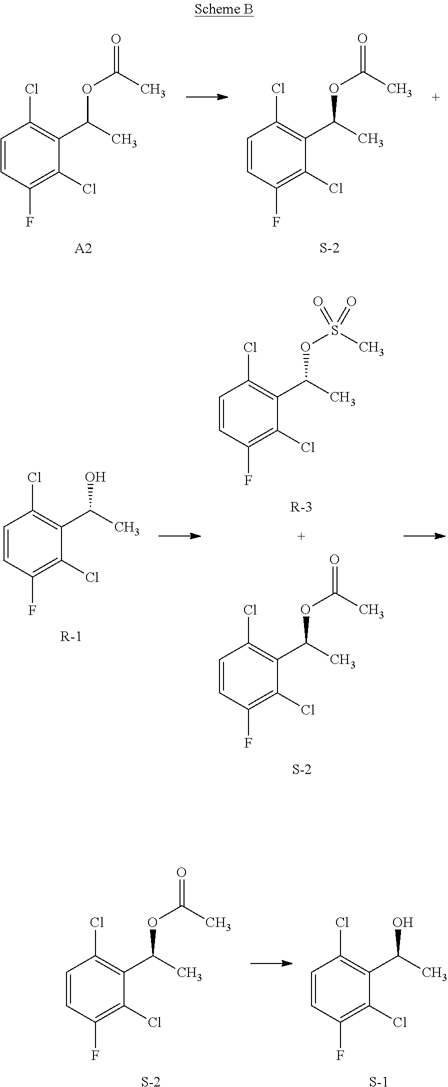

To a 50 mL jacketed flask equipped with a pH electrode, an overhead stirrer and a base addition line (1 M NaOH), was added 1.2 mL of 100 mM potassium phosphate buffer pH 7.0 and 0.13 mL of PLE AS suspension. Then, compound A2 (0.13 g, 0.5 mmol, 1.00 eq) was added dropwise and the resulting mixture was stirred at room temperature for 20 h, maintaining the pH of the reaction constant at 7.0 using 1 M NaOH. Both the conversion and enantiomeric excesses (ee's) of the reaction were monitored by RP-HPLC, and stopped after 50% starting material was consumed (approximately 17 hours under these conditions). The mixture was then extracted three times with 10 mL of ethyl acetate to recover both ester and alcohol as a mixture of R-1 and S-2.

Methanesulfonyl chloride (0.06 mL, 0.6 mmol) was added to a solution of a mixture of R-1 and S-2 (0.48 mmol) in 4 mL of pyridine under nitrogen atmosphere. The reaction mixture was stirred at room temperature for 3 h then evaporated to obtain an oil. Water (20 mL) was added to the mixture and then EtOAc (20 mL×2) was added to extract the aqueous solution. The organic layers were combined, dried, filtered, and evaporated to give a mixture of R-3 and S-2. This mixture was used in the next step reaction without further purification. ¹H NMR (400 MHz, chloroform-D) δ ppm 1.66 (d, J=7.1 Hz, 3H) 1.84 (d, J=7.1 Hz, 3H) 2.09 (s, 3H) 2.92 (s, 3H) 6.39 (q, J=7.0 Hz, 1H) 6.46 (q, J=6.8 Hz, 1H) 6.98-7.07 (m, 1H) 7.07-7.17 (m, 1H) 7.23-7.30 (m, 1H) 7.34 (dd, J=8.8, 4.80 Hz, 1H).

Potassium acetate (0.027 g, 0.26 mmol) was added to a mixture of R-3 and S-2 (0.48 mmol) in 4 mL of DMF under nitrogen atmosphere. The reaction mixture was heated to 100° C. for 12 h. Water (20 mL) was added to the reaction mixture and EtOAc (20 mL×2) was added to extract the aqueous solution. The combined organic layer was dried, filtered, and evaporated to give an oil of S-2 (72 mg, 61% yield in two steps). Chirality ee: 97.6%. ¹H NMR (400 MHz, chloroform-D) δ ppm 1.66 (d, J=7.1 Hz, 3H) 2.09 (s, 3H) 6.39 (q, J=6.8 Hz, 1H) 7.02 (t, J=8.5 Hz, 1H) 7.22-7.30 (m, 1H).

Sodium methoxide (19 mmol; 0.5 M in methanol) was added slowly to compound S-2 (4.64 g, 18.8 mmol) under a nitrogen atmosphere at 0° C. The resulting mixture was stirred at room temperature for 4 hours. The solvent was evaporated and H₂O (100 mL) was added. The cooled reaction mixture was neutralized with sodium acetate-acetic acid buffer solution to pH 7. Ethyl acetate (100 mL×2) was added to extract the aqueous solution. The combined organic layers were dried over Na₂SO₄, filtered, and evaporated to obtain S-1 as a white solid (4.36 g, 94.9% yield); SFC-MS: 97% ee. ¹H NMR (400 MHz, chloroform-D) δ ppm 1.65 (d, J=6.8 Hz, 3H) 5.58 (q, J=6.9 Hz, 1H) 6.96-7.10 (m, 1H) 7.22-7.36 (m, 1H).

### 5-bromo-3-[1-(2,6-dichloro-3-fluoro-phenyl)-ethoxy]-pyridin-2-ylamine (racemate)

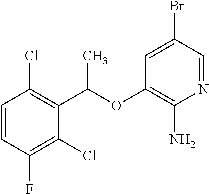 [SMILES: CC(Oc1cc(Br)cnc1N)c1c(Cl)ccc(F)c1Cl]

1. 2,6-Dichloro-3-fluoroacetophenone (15 g, 0.072 mol) was stirred in THF (150 mL, 0.5M) at 0° C. using an ice bath for 10 min. Lithium aluminum hydride (2.75 g, 0.072 mol) was slowly added. The reaction was stirred at ambient temperature for 3 hr. The reaction was cooled in ice bath, and water (3 mL) was added drop wisely followed by adding 15% NaOH (3 mL) slowly. The mixture was stirred at ambient temperature for 30 min. 15% NaOH (9 mL), MgSO₄ were added and the mixture filtered to remove solids. The solids were washed with THF (50 mL) and the filtrate was concentrated to give 1-(2,6-dichloro-3-fluoro-phenyl)-ethanol (14.8 gm, 95% yield) as a yellow oil. ¹H NMR (400 MHz, DMSO-d₆) δ 1.45 (d, 3H), 5.42 (m, 2H), 7.32 (m, 1H), 7.42 (m, 1H).

2. To a stirred solution of triphenyl phosphine (8.2 g, 0.03 mol) and DEAD (13.65 mL of a 40% solution in toluene) in THF (200 mL) at 0° C. was added a solution of 1-(2,6-dichloro-3-fluoro-phenyl)-ethanol (4.55 g, 0.021 mol) and 3-hydroxy-nitropyridine (3.35 g, 0.023 mol) in THF (200 mL). The resulting bright orange solution was stirred under a nitrogen atmosphere at ambient temperature for 4 hours at which point all starting materials had been consumed. The solvent was removed, and the crude material was dry loaded onto silica gel, and eluted with ethyl acetate-hexanes (20:80) to yield 3-(2,6-dichloro-3-fluoro-benzyloxy)-2-nitro-pyridine (6.21 g, 0.021 mol, 98%) as a pink solid. ¹H NMR (CDCl₃, 300 MHz) δ1.8-1.85 (d, 3H), 6.0-6.15 (q, 1H), 7.0-7.1 (t, 1H), 7.2-7.21 (d, 1H), 7.25-7.5 (m, 2H), 8.0-8.05 (d, 1H).

3. To a stirred mixture of AcOH (650 mL) and EtOH (500 mL) was suspended 3-(2,6-dichloro-3-fluoro-benzyloxy)-2-nitro-pyridine (9.43 g, 0.028 mol) and iron chips (15.7 g, 0.28 mol). The reaction was heated slowly to reflux and allowed to stir for 1 hr. The reaction was cooled to room temperature then diethyl ether (500 mL) and water (500 mL) was added. The solution was carefully neutralized by the addition of sodium carbonate. The combined organic extracts were washed with sat'd NaHCO₃ (2×100 mL), H₂O (2×100 mL) and brine (1×100 mL) then dried (Na₂SO₄), filtered and concentrated to dryness under vacuum to yield 3-(2,6-dichloro-3-fluoro-benzyloxy)-pyridin-2-ylamine (9.04 g, 0.027 mol, 99%) as a light pink solid. ¹H NMR (CDCl₃, 300 MHz) δ1.8-1.85 (d, 3H), 4.9-5.2 (brs, 2H), 6.7-6.84 (q, 1H), 7.0-7.1 (m, 1H), 7.2-7.3 (m, 1H), 7.6-7.7 (m, 1H).

4. A stirring solution of 3-(2,6-dichloro-3-fluoro-benzyloxy)-pyridin-2-ylamine (9.07 g, 0.03 mol) in acetonitrile was cooled to 0° C. using an ice bath. To this solution was added N-bromosuccinimide (NBS) (5.33 g, 0.03 mol) portionwise. The reaction was stirred at 0° C. for 15 min. The reaction was concentrated to dryness under vacuum. The resulting dark oil was dissolved in EtOAc (500 mL), and purified via silica gel chromatography. The solvents were then removed in vacuo to yield 5-bromo-3-(2,6-dichloro-3-fluoro-benzyloxy)-pyridin-2-ylamine (5.8 g, 0.015 mol, 51%) as a white crystalline solid. ¹H NMR (CDCl₃, 300 MHz) δ1.85-1.95 (d, 3H), 4.7-5.0 (brs, 2H), 5.9-6.01 (q, 1H), 6.8-6.95 (d, 1H), 7.01-7.2 (t, 1H), 7.4-7.45 (m, 1H), 7.8-7.85 (d, 1H).

### 5-bromo-3-[1 (R)-(2,6-dichloro-3-fluoro-phenyl)-ethoxy]-pyridin-2-ylamine

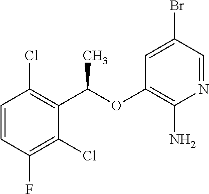 [SMILES: C[C@@H](Oc1cc(Br)cnc1N)c1c(Cl)ccc(F)c1Cl]

The enantiomerically pure R isomer was prepared as described above for the racemate, but using the enantiomerically pure starting materials described above. ¹H NMR (400 MHz, DMSO-d₆) δ 1.74 (d, 3H), 6.40 (m, 1H), 6.52 (br s, 2H), 7.30 (m, 1H), 7.48 (m, 1H), 7.56 (s, 1H); MS m/z 382 (M+1).

### 4-methanesulfonyloxy-piperidine-1-carboxylic acid tert-butyl ester (2)

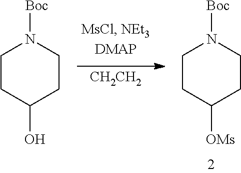

To a stirred solution of 4-hydroxy-piperidine-1-carboxylic acid tert-butyl ester (7.94 g, 39.45 mmol) in CH₂Cl₂ (100 mL), cooled to 0° C., was slowly added NEt₃ (5.54 mL, 39.45 mmol) followed by methane sulfonyl chloride (3.06 mL, 39.45 mmol) and DMAP (48 mg, 0.39 mmol). The mixture was stirred at room temperature overnight. To the mixture was added water (30 mL). Extraction with CH₂Cl₂ (3×30 mL) followed by drying (Na₂SO₄) and removal of the solvent in vacuo afforded 4-methanesulfonyloxy-piperidine-1-carboxylic acid tert-butyl ester as a white solid (11.00 g, >99% yield). ¹H NMR (CDCl₃, 400 MHz) δ 4.89 (m, 1H), 3.69 (m, 2H), 3.31 (m, 2H), 3.04 (s, 3H), 1.95 (m, 2H), 1.83 (m, 2H), 1.46 (s, 9H).

### tert-butyl-4-[4-(4,4,5,5-tetramethyl-1,3,2-dioxaborolan-2-yl)-1H-pyrazol-1-yl]biperidine-1-carboxylate

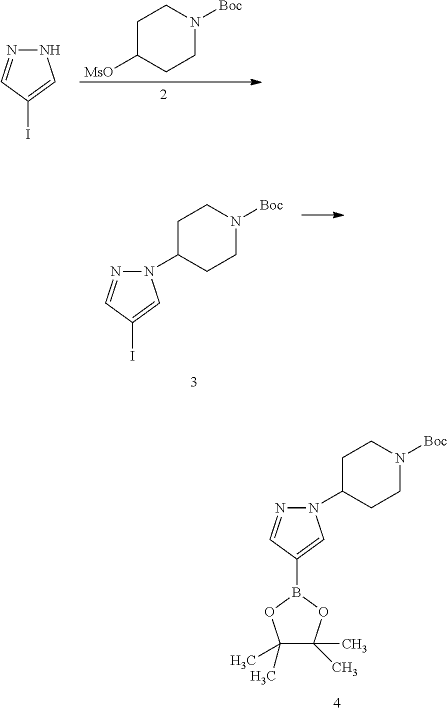 [SMILES: Ic1cn[nH]c1 ; CC(C)(C)OC(=O)N1CCC(OS(C)(=O)=O)CC1 ; CC(C)(C)OC(=O)N1CCC(n2cc(I)cn2)CC1 ; CC(C)(C)OC(=O)N1CCC(n2cc(B3OC(C)(C)C(C)(C)O3)cn2)CC1]

### tert-butyl 4-(4-iodo-1H-pyrazol-1-yl)piperidine-1-carboxylate (3)

NaH (1.2 eq., 0.68 mmol) was added portionwise to a stirred solution of 4-iodopyrazole (0.57 mmol) in DMF (2 L) at 4° C. The resulting mixture was stirred for 1 hour at 4° C. and 4-methanesulfonyloxy-piperidine-1-carboxylic acid tert-butyl ester, compound 2 (1.1 eq., 0.63 mmol) was then added. The resulting mixture was heated to 100° C. for 12 h. The reaction was quenched with H₂O and extracted with EtOAc several times. The combined organic layers were dried, filtered, and concentrated to afford an orange oil. The residue was purified by silica gel chromatography (eluting with 5% EtOAc in pentane) to give compound 3 as a white solid (140 g, 66%).

### tert-butyl-4-[4-(4,4,5,5-tetramethyl-1,3,2-dioxaborolan-2-yl)-1H-pyrazol-1-yl]biperidine-1-carboxylate (4)

Bis(pinacolato)diboron (1.4 eq., 134 g, 0.52 mol) and potassium acetate (4 eq., 145 g, 1.48 mol) were added sequentially to a solution of compound 3 (140 g, 0.37 mol) in 1.5 L of DMSO. The mixture was purged with nitrogen several times and dichlorobis(triphenylphosphino) palladium (II) (0.05 eq., 12.9 g, 0.018 mol) was then added. The resulting mixture was heated at 80° C. for 2 h. The reaction mixture was cooled to room temperature and filtered through a bed of Celite® and washed with EtOAc. The filtrate was washed with saturated NaCl (500 mL×2), dried over Na₂SO₄, filtered and concentrated. The residue was purified by silica gel chromatography (eluting with 5% EtOAc in hexanes) to give compound 4 as a white solid (55 g, 40%).

### 3-[(R)-1-(2,6-dichloro-3-fluoro-phenyl)-ethoxy]-5-(1-piperidin-4-yl-1H-pyrazol-4-yl)-pyridin-2-ylamine (1)

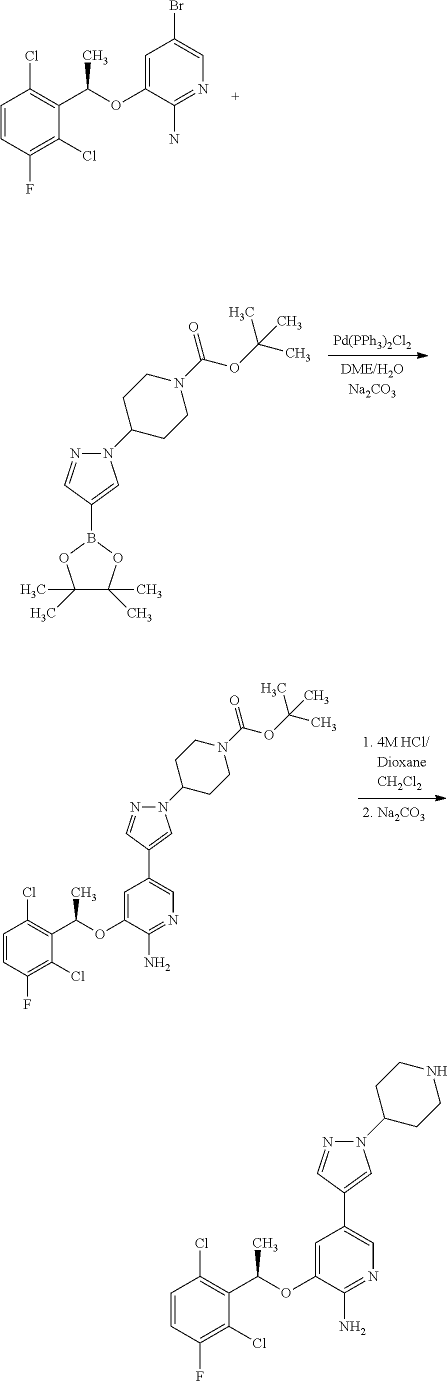 [SMILES: C[C@@H](Oc1cc(Br)cnc1N)c1c(Cl)ccc(F)c1Cl ; CC(C)(C)OC(=O)N1CCC(n2cc(B3OC(C)(C)C(C)(C)O3)cn2)CC1 ; C[C@@H](Oc1cc(-c2cnn(C3CCN(C(=O)OC(C)(C)C)CC3)c2)cnc1N)c1c(Cl)ccc(F)c1Cl ; C[C@@H](Oc1cc(-c2cnn(C3CCNCC3)c2)cnc1N)c1c(Cl)ccc(F)c1Cl ; "Pd(PPh3)2Cl2"]

To a stirred solution of 3-[(R)-1-(2,6-dichloro-3-fluoro-phenyl)-ethoxy]-5-(4,4,5,5-tetramethyl-[1,3,2]dioxaborolan-2-yl)-pyridin-2-ylamine (15.22 g, 35.64 mmol) and 4-(4-bromo-pyrazol-1-yl)-piperidine-1-carboxylic acid tert-butyl ester (14.12 g, 42.77 mmol) in DME (143 mL) was added a solution of Na₂CO₃ (11.33 g, 10692 mmol) in water (36 mL). The solution was degassed and charged with nitrogen three times. To the solution was added Pd(PPh₃)₂Cl₂ (1.25 mg, 1.782 mmol). The reaction solution was degassed and charged with nitrogen again three times. The reaction solution was stirred at 87° C. oil bath for about 16 hours (or until consumption of the borane pinacol ester), cooled to ambient temperature and diluted with EtOAc (600 mL). The reaction mixture was filtered through a pad of Celite® and washed with EtOAc. The EtOAc solution was washed with brine, dried over Na₂SO₄, and concentrated. The crude product was purified on a silica gel column eluting with EtOAc/Hexane system (Biotage 90+ Column: equilibrium 600 mL 100% Hexanes, segment 1: 2250 mL 50% EtOAc/Hexanes Linear, segment 2: 4500 mL 75% EtOAc/Hexanes Linear, segment 3: 4500 mL 100% EtOAc) to afford 4-(4-{6-amino-5-[(R)-1-(2,6-dichloro-3-fluoro-phenyl)-ethoxy]-pyridin-3-yl}-pyrazol-1-yl)-piperidine-1-carboxylic acid tert-butyl ester (11.8 g, 60% yield, ˜95% purity) with a Rf of 0.15 (50% EtOAc/Hexanes). MS m/e 550 (M+1)⁺.

To a solution of 4-(4-{6-amino-5-[(R)-1-(2,6-dichloro-3-fluoro-phenyl)-ethoxy]-pyridin-3-yl}-pyrazol-1-yl)-piperidine-1-carboxylic acid tert-butyl ester (11.8 g, 21.45 mmol) in CH₂Cl₂ (59 mL, 0.2M) was added 4N HCl/Dioxane (21 mL). The solution was stirred overnight forming a solid. The solid was crushed thoroughly with a glass rod and sonicated to release starting material trapped in the solid. Additional 4N HCl/Dioxane (21 mL) was added and stirred for another 2 hours at room temperature in which LCMS showed no starting material. The suspension was filtered in a Buchner funnel lined with filter paper. The mother liquor was saved because it contained <5% of product. The solid was transferred to a 500 mL beaker and HPLC water was added until the solid dissolved completely. The pH was adjusted to 10 with the addition of solid Na₂CO₃. The water solution was extracted with CH₂Cl₂ (5×200 mL) or until LCMS showed no product in the aqueous layer. The CH₂Cl₂ solution was dried over Na₂SO₄ and concentrated. The crude product, re-dissolved in CH₂Cl₂ (10 mL) and MeOH (1 mL), was purified on a silica gel column eluting with CH₂Cl₂/MeoH/NEt₃ system (Biotage 40+Column: equilibrium 600 mL CH₂Cl₂ 100% giving byproduct, segment 1: 1200 mL 10% MeOH/CH₂Cl₂ linear, segment 2: 2400 mL 10% MeOH/CH₂Cl₂ step, segment 3: 2400 mL 9% MeOH/1% NEt₃/CH₂Cl₂). The desired fractions were collected to provide 3-[(R)-1-(2,6-dichloro-3-fluoro-phenyl)-ethoxy]-5-(1-piperidin-4-yl-1H-pyrazol-4-yl)-pyridin-2-ylamine (7.19 g, 75% combined yield, white solid). MS m/e 450 (M+1)⁺. ¹H NMR (DMSO-d₆, 400 MHz) δ 7.92 (s, 1H), 7.76 (s, 1H), 7.58 (m, 1H), 7.53 (s, 1H), 7.45 (m, 1H), 6.90 (s, 1H), 6.10 (m, 1H), 5.55 (bs, 2H), 4.14 (m, 1H), 3.05 (m, 2H), 2.58 (m, 2H), 1.94 (m, 2H), 1.80 (d, 3H), 1.76 (m, 2H).

The solid product 3-[(R)-1-(2,6-dichloro-3-fluoro-phenyl)-ethoxy]-5-(1-piperidin-4-yl-1H-pyrazol-4-yl)-pyridin-2-ylamine was dissolved in dichloromethane, and the solvent was evaporated slowly to generate fine crystalline solid. After high vacuum dry, the sample was confirmed to be a single crystalline polymorph form A with a melting point of 194° C.

1. A method of treating cancer in a mammal comprising administering to said mammal a therapeutically effective amount of 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof, wherein the cancer is mediated by at least one genetically altered ROS.

2. The method of claim 1, wherein the mammal is a human.

3. The method of claim 1, wherein the at least one genetically altered ROS is a genetically altered ROS gene.

4. The method of claim 3, wherein the genetically altered ROS gene is a ROS fusion gene.

5. The method of claim 4, wherein the ROS fusion gene is the SLC34A2-ROS gene or the CD74-ROS gene

6. The method of claim 4, wherein the ROS fusion gene is the FIG-ROS gene.

7. The method of claim 1, wherein the at least one genetically altered ROS is a ROS fusion protein.

8. The method of claim 7, wherein the ROS fusion protein is SLC34A2-ROS kinase.

9. The method of claim 7, wherein the ROS fusion protein is CD74-ROS kinase.

10. The method of claim 7, wherein the ROS fusion protein is FIG-ROS kinase.

11. The method of claim 1, wherein the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

12. The method of claim 1, wherein the cancer is selected from the group consisting of non-small cell lung cancer (NSCLC), glioblastoma, squamous cell carcinoma, hormone-refractory prostate cancer, papillary renal cell carcinoma, colorectal adenocarcinoma, neuroblastomas, anaplastic large cell lymphoma (ALCL) and gastric cancer.

13. The method of claim 1, wherein the cancer is non-small cell lung cancer (NSCLC).

14. The method of claim 1, wherein the cancer is glioblastoma.

15. The method of claim 1, wherein 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof is administered as a pharmaceutical composition comprising 3-[(R)-1-(2,6-dichloro-3-fluoro-phenyl)-ethoxy]-5-(1-piperidin-4-yl-1H-pyrazol-4-yl)-pyridin-2-ylamine or a pharmaceutically acceptable salt thereof and at least one pharmaceutically acceptable carrier.
